(19) United States
(12) Patent Application Publication
(10) Pub. No.: US 2011/0288484 A1
(43) Pub. Date: Nov. 24, 2011

(54) METHOD AND ASSOCIATED APPARATUS FOR COATING PROJECTIONS ON A PATCH ASSEMBLY

(75) Inventors: Mark Anthony Fernance Kendall, Chelmer (AU); Germain Jitendra Fernando, Indooroopilly (AU); Xianfeng Chen, Toowong (AU); Tarl Prow, Collingwood Park (AU); Holly Corbett, Dutton Park (NZ)

(73) Assignee: The Univeristy of Queensland

(21) Appl. No.: 13/124,109

(22) PCT Filed: Oct. 16, 2009
(86) PCT No.: PCT/AU2009/001366

§ 371 Date: Aug. 8, 2011

(30) Foreign Application Priority Data
Oct. 16, 2008 (AU) 2008905364

(51) Int. Cl. A61M 5/00; B05C 11/00; C25D 13/12; B05D 3/12

(52) U.S. Cl. 604/173; 427/2.28; 118/423; 118/708; 204/471

(57) ABSTRACT

A method of coating projections on a patch, the method including, selecting a coating solution viscosity, the viscosity being selected to reduce the degree of capillary action between the patch and the coating solution and immersing at least part of tips of projections in a coating solution having the selected coating solution viscosity such that substantially only tips of the projections are coated.

### BACKGROUND OF THE INVENTION

The present invention relates to a method and apparatus for coating projections on a patch and in particular to a method and apparatus for coating projection tips.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Needle and syringe are the most common method to deliver vaccines to humans. It is estimated by World Health Organization (WHO) that 600 million injections are given annually for immunisations. However, this method has several limitations. First of all, needle phobia makes immunisations stressful. Second, accidental needle-stick injuries happen in both developed and developing countries; for example, around 300,000 needle-stick injuries occur annually in US hospitals alone. Third, a great number of infections are caused by the improper and unsafe use of needle and syringe. Last but most importantly, using needle and syringe is not able to efficiently deliver the payload to the densely packed cells in the outer skin layer needed for inducing immune responses.

To overcome these problems, many approaches have been developed, such as, diffusion/permeation delivery, liquid jet injection, biolistic microparticle injection, etc. However, these approaches still have disadvantages. For example, diffusion/permeation delivery can only be used for small molecules; liquid jet injection is not pain-free and needs address the challenge of more accurate delivery; biolistic microparticle injection is difficult to achieve accurate targeting of specific cells due to the tough outer skin layer (the stratum corenum).

It is known to provide patches including a number of projections thereon to allow bioactive material to be administered to a subject. Such arrays of projections or needles on a patch are an increasingly effective way of delivering therapeutic agents or biomarkers since there is minimal or no pain, little or no injury from the needle and highly reduced possibility of cross infection. The solid projections or needles on a patch can be coated with drugs or macromolecules. These can be subsequently delivered to a desired target by the penetration of the projections or needles into the skin.

For example, WO2005/072630 describes devices for delivering bioactive materials and other stimuli to living cells, methods of manufacture of the device and various uses of the device, including a number of medical applications. The device comprises a plurality of projections which can penetrate a body surface so as to deliver the bioactive material or stimulus to the required site. The projections are typically solid and the delivery end section of the projection is so dimensioned as to be capable of insertion into targeted cells to deliver the bioactive material or stimulus without appreciable damage to the targeted cells or specific sites therein.

The microprojections reported in literature are relatively large and sparsely packed. The length is generally 200 to 700 μm and the density is less than 321 projections per cm².

Various methods of coating patches are also known. For example, microneedle arrays can be coated with a drug by immersion in aqueous formulations containing drug and polysorbate 20 (Michel Cormier, Bonny Johnson, Mahmoud Ameri, Kofi Nyam, Luz Libiran, Dee Dee Zhang, Pete Daddona, Journal of Controlled Release 97 (2004) 503-511). Microprojection arrays are also known to be coated by immersion in an aqueous solution of ovabulmin (OVA) (James A. Matriano, Michel Cormier, Juanita Johnson, Wendy A. Young, Margaret Buttery, Kofi Nyam, and Peter E. Daddona, Pharmaceutical Research, 19 (2002) 63-70). The arrays were air-dried for 1 h at ambient conditions. The length of each microprojection is 330 μm. The density of projections is 190 projections/cm². However, the substrate that carries the microprojection arrays is contaminated in these coating processes.

WO02/074173 and U.S. Pat. No. 6,855,372 describe an apparatus and method for selectively applying an agent-containing liquid coating to skin piercing microprojections. The coating solution is applied to the skin piercing microprojections by providing an agent-containing coating liquid and by moving the microprojections tangentially across and through a thin film of the liquid provided on a rotating drum. However, this technique has a tendency to result in ripple formations in the film while dipping microprojections. The ripples cause liquid to touch and coat the substrate that carries the microprojections and cause differences in coating length of microprojections on the leading and trailing edge of the array. This is restricted to certain dip lengths and to certain spacing between microprojections, given that wicking of liquid up between closely spaced microprojections can cause unwanted coating of the base. It therefore would be desirable to provide microprojection coating processes that reduces or eliminates between-needle wicking and offers better coating uniformity and better control of dip/coating length on each microprojection. It would also be desirable to provide improved methods for precisely coating microprojections or other microstructures with a variety of materials, including materials other than homogeneous liquid solutions.

Microprojection arrays are also known to be coated by being dipped into a coating solution reservoir through dip-holes at the same spacing as the microneedles in the array (Harvinder S. Gill and Mark R Prausnitz, Journal of Controlled Release, 117 (2007) 227-237 and Harvinder S. Gill and Mark R Prausnitz, Pharmaceutical Research, 24 (2007) 1369-1380). The diameter of the “dip-holes” is twice the width of microprojections for misalignment tolerance. The coating solution contains carboxymethylcellulose (CMC) sodium salt, poloxamer 188 and a suitable drug. The size of the projection is around 700 μm in length, 160 μm in width and 50 μm in thickness. The distance between projections is over a few mm. However, this method is unsuitable for many patches due to difficulties in aligning the projections with the dip-holes, particularly on patches with densely packed, shorter projections.

Therefore, it would be desirable to provide a simple method that offers great coating uniformity and control of dip/coating on each microprojection but does not require the use of a physical mask. Currently, all compounds have to be dissolved in a coating solution before coating; however, there are numerous biopharmaceuticals that are not soluble. Hence, it also would be desirable to provide a method that can coat insoluble compounds.

In summary, the previous systems have focussed on coating large and very sparsely packed projections. Such techniques often prove to be unsuccessful or difficult when coating small and densely packed projections.

For successful vaccine delivery systems, effective dry coating of the vaccine only on the patch projections in a controlled manner, followed by the rapid, subsequent release of an effective amount of the vaccine after application of the patch, is desired. Further, whilst it is desirable to employ patches that have smaller projections or needles, effectively coating these using existing techniques is difficult.

### SUMMARY OF THE PRESENT INVENTION

The present invention seeks to substantially overcome, or at least ameliorate, one or more disadvantages of existing arrangements.

In a first broad form the present invention seeks to provide a method of coating projections on a patch, the method including:
a) selecting a coating solution viscosity, the viscosity being selected to reduce the degree of capillary action between the patch and the coating solution; and,
b) immersing at least part of tips of projections in a coating solution having the selected coating solution viscosity such that substantially only tips of the projections are coated.

Typically the coating solution viscosity is at least one of:
a) 1 Pa.S;
b) 10 Pa.S; and,
c) 50 Pa.S.

Typically the method includes:
a) selecting the coating solution viscosity in accordance with an immersion time; and,
b) immersing at least part of the tips of the projections for the immersion time.

Typically the immersion time is less than at least one of:
a) 60 minutes;
b) 10 minutes;
c) 1 minute; and,
d) 10 seconds.

Typically the method includes drying the coated projection tips.

Typically the method includes drying the coated projection tips using at least one of:
a) exposure to vacuum;
b) temperature control;
c) humidity control;
d) a gas flow.

Typically viscosity is selected in accordance with patch properties including at least one of:
a) projection size;
b) projection shape; and,
c) projection spacing.

Typically the viscosity is selected in accordance with a contact angle representing hydrophilicity or hydrophobicity of the patch.

Typically the method includes modifying the surface properties of the patch to thereby control at least one of:
a) hydrophilicity of the patch;
b) hydrophobocity of the patch; and,
c) wettability of the patch.

Typically the method includes modifying the surface properties of the patch prior to immersing the tips.

Typically the method includes modifying the surface properties of the patch by modifying a surface structure of at least part of the patch.

Typically the surface structure includes a surface roughness.

Typically the method includes modifying the surface structure by at least one of:
a) mechanical means; and,
b) chemical means.

Typically the method includes modifying the surface properties of the patch by coating the patch.

Typically the method includes coating the patch with at least one of:
a) 3-aminopropyl triethoxysilane (3-APTES) solution; and,
b) Methylcellulose.

Typically the method includes selecting a coating solution surface tension.

Typically the method includes selecting at least the viscosity to thereby control an amount of coating on the tips.

Typically the coating solution includes a material that is insoluble in the coating solution and wherein the material is distributed substantially homogenously throughout the coating solution.

Typically the material is at least one of:
a) a biological agent; and,
b) a therapeutic agent.

Typically the material is at least one of:
a) nanoparticles;
b) a nucleic acid or protein;
c) an antigen, allergen, or adjuvant;
d) parasites, bacteria, viruses, or virus-like particles;
e) quantum dots, SERS tags, raman tags or other nanobiosensors;
f) metals or metallic compounds;
g) molecules, elements or compounds;
h) DNA having a concentration of between 0.01 mg/ml and 5 mg/ml; and,
i) protein having a concentration of between 0.01 mg/ml and 50 mg/ml

Typically the coating solution includes at least one of:
a) a viscosity enhancer;
b) a detergent;
c) a surfactant; and,
d) an adjuvant.

Typically the adjuvant acts as a detergent.

Typically at least one of:
a) the viscosity enhancer is 0% to 90% of the coating solution; and,
b) the detergent is 0% to 90% of the coating solution.

Typically the viscosity enhancer is at least one of:
a) honey;
b) pectin;
c) methylcellulose;
d) carboxymethylcellulose (CMC);
e) sodium alginate;
f) gelatine;
g) agar; and,
h) agarose.

Typically the method includes:
a) applying an electrical signal to the coating solution and the projections; and,
b) controlling the coating process using the electrical signal.

Typically the method includes applying an electrical signal to the coating solution and the projections to thereby attract a material within the coating solution onto the projections using electrophoresis.

Typically the method includes controlling a length of coating on the projections by controlling a depth of immersion in the coating solution.

Typically the method includes controlling a depth of immersion in the coating solution based on a coating solution depth.

Typically the method includes immersing the tips by placing at least the projections in a well containing the coating solution.

Typically the well includes a stop, and wherein the stop cooperates with the patch such that only projection tips are immersed in the coating solution.

Typically the stop abuts against a patch base.

Typically the projection tips abut against a floor of the well.

Typically the method includes coating the projections a number of times.

Typically the method includes:
a) coating the surface a first time using a first set of coating parameters; and,
b) coating the surface at least a second time using a second set of coating parameters different to the first set of coating parameters.

Typically the method includes coating the projections a number of times to thereby provide at least first and second coating layers.

Typically the second layer overlays the first layer to thereby protect the first layer during insertion into the subject.

Typically the first and second layers include different coating materials.

Typically the method includes coating a first length of the projection using a first coating material and a second length of the projection using a second coating material.

Typically the first and second coating lengths are selected to deliver material to a selected region in a subject.

Typically the projections are solid.

Typically the projections are non-porous and non-hollow.

In a second broad form the present invention seeks to provide a method of coating projections on a patch, the method including immersing at least part of tips of projections in a coating solution, the coating solution having a viscosity of greater than approximately 1 Pa.S.

In a third broad form the present invention seeks to provide apparatus for use in coating projections on a patch, the apparatus including a positioning system for immersing at least part of tips of projections in a coating solution, the coating solution having a viscosity selected in accordance with patch properties so as to reduce the degree of capillary action between the patch and the coating solution such that substantially only tips of the projections are coated.

Typically the positioning system includes a support having an arm for supporting the patch relative to the coating solution.

Typically the arm is movable to thereby allow the relative position of the patch and the coating solution to be controlled.

Typically the positioning system includes a movable platform for supporting the coating solution to thereby allow the relative position of the patch and the coating solution to be controlled.

Typically the apparatus includes a controller for controlling the positioning system.

Typically the controller is coupled to a sensor for determining at least if the projection tips are immersed.

Typically the sensor is an imaging system for imaging the projections and the coating solution.

Typically the sensor includes a signal generator for:
a) applying an electrical signal to the projections and the coating solution; and,
b) providing an indication relating to the signal to the controller, thereby allowing the controller to determine if the projection tips are immersed.

Typically the apparatus includes a signal generator for applying an electrical signal to the projections and the coating solution to thereby attract a material within the coating solution onto the projections using electrophoresis.

Typically the apparatus is for controlling a length of coating on the projections by controlling a depth of immersion in the coating solution.

Typically the method includes controlling a depth of immersion in the coating solution based on a coating solution depth.

Typically the apparatus includes a well containing the coating solution.

Typically the positioning system includes a stop provided on the well, and wherein the stop cooperates with the patch such that only projection tips are immersed in the coating solution.

Typically the stop abuts against a patch base.

Typically the positioning system includes the depth of coating solution in the well so that the projection tips abut against a floor of the well when the projection tips are immersed.

Typically the apparatus includes a force sensitive device for detecting if the projections are in contact with the well.

Typically a length of coating on the projections is controlled at least in part by a depth of coating solution in the well.

Typically the apparatus is for agitating the patch relative to the coating solution.

Typically the apparatus agitates the patch relative to the coating solution using at least one of:
a) an arm; and,
b) a movable platform

Typically the apparatus is for oscillating the patch relative to the coating solution.

Typically the apparatus includes a controller for controlling at least one of an amplitude and frequency of the oscillation.

Typically at least one of:
a) the amplitude of the oscillation is in the range of 0.01 to 100 μm; and,
b) the frequency of the oscillation is in the range of 1 to 10,000 Hz.

Typically at least one of:
a) the amplitude of the oscillation is approximately ±1 μm; and,
b) the frequency of the oscillation is approximately 400 Hz.

In a fourth broad form the present invention seeks to provide a patch for delivering material to a subject, the patch including a number of projections thereon, the projections being coated by immersion of at least part of the projections in a coating solution having a viscosity selected to reduce the degree of capillary action between the patch and the coating solution.

Typically the coating solution has a viscosity of at least:
a) 1 Pa.S;
b) 10 Pa.S; and,
c) 50 Pa.S.

It will be appreciated that the broad forms of the invention can be used independently or in conjunction, depending on the preferred implementation.

### BRIEF DESCRIPTION OF THE DRAWINGS

An example of the present invention will now be described with reference to the accompanying drawings, in which:

FIGS. 1A and 1B are schematic side and plan views of an example of device for delivery of material to targets within a body;

FIG. 1C is a schematic diagram of an example of the device of FIG. 1A in use;

FIGS. 1D to 1F are schematic diagrams of examples of projections used in the device of FIG. 1A;

FIG. 2 is a schematic diagram illustrating the transfer of coating material to a subject, in use;

FIGS. 3A and 3B are schematic diagrams of examples of apparatus for use in coating projection tips;

FIG. 3C and 3D are schematic diagrams of an example of a mould having a number of wells;

FIG. 3E is a schematic diagram of an example of apparatus for positioning projection tips in coating solution;

FIGS. 3F to 3I are schematic diagrams of an example of the immersion and withdrawal of projection tips into coating solution;

FIG. 3J is a schematic diagram of an example of apparatus for automatically positioning projection tips in coating solution;

FIGS. 4A and 4B are secondary electron images of examples of projections;

FIGS. 5A to 5F are examples of secondary electron images of patches coated using different low viscosity coating solutions;

FIGS. 6A and 6B are examples of secondary electron images of patches coated using a high viscosity coating solution;

FIGS. 7A to 7D are secondary electron images of examples of patches including the projections of FIG. 4A coated using a high viscosity coating gel;

FIG. 8A is an example of a fluorescence image of rhodamine-dextran coating on projection tips;

FIG. 8B is an example of a fluorescence image of fluorescent dye released from coated projection tips in the ear skin of a C57BL/6 mouse;

FIGS. 9A to 9D are secondary electron images of examples of patches including the projections of FIG. 4B coated using a high viscosity coating gel;

FIG. 10 is an example of secondary electron image of an example of a patch including the projections of FIG. 4B coated using a high viscosity coating gel;

FIGS. 11A to 11D are secondary electron images of examples of coated projection tips before and after being applied mouse skin;

FIG. 11E is a fluorescence image of an example of coated projection tips before being applied mouse skin;

FIG. 11F is a fluorescence image of an example of coated projection tips co-localised with antigen presenting cells in mouse skin;

FIG. 12A is a schematic diagram of an example of a single projection coated with multiple layers;

FIG. 12B is an example of a Multi-Photon Microscope image showing fluorescence from FITC-Dextran and Rhodamine-Dextran on coated projections;

FIG. 12C is a secondary electron image of a patch coated with multiple layers of FITC-Dextran and Rhodamine-Dextran; and,

FIGS. 12D and 12E are examples of Multi-Photon Microscope images demonstrating the delivery of FITC-Dextran and Rhodamine-Dextran to a mouse ear.

FIG. 13A is a schematic diagram showing patch surface modification by APTES (3-aminopropyl triethoxysilane) solution;

FIG. 13B is a secondary electron image of an example of an untreated patch coated using sodium alginate;

FIG. 13C is a secondary electron image of an example of a treated patch coated using a coating solution containing sodium alginate;

FIG. 13D is a secondary electron image of an example of a treated patch coated multiple times using a coating solution containing sodium alginate;

FIG. 13E is a backscattered electron image of the coated patch of FIG. 13D;

FIG. 14 is a graph of an example of the immune responses induced by influenza vaccine delivered by patches and intramuscular injection;

FIG. 15 is a graph showing an example of the normalized release of ¹⁴C OVA into mouse ear skin following different times of patch application;

FIG. 16A is a schematic diagram of a first example of a single projection coated with multiple layers;

FIGS. 16B and 16C are schematic diagrams of an example of the delivery of material to a subject using the projection of FIG. 16A;

FIG. 16D is a schematic diagram of a first example of a single projection coated with multiple layers; and,

FIGS. 16E and 16F are schematic diagrams of an example of the delivery of material to a subject using the projection of FIG. 16D.

### DETAILED DESCRIPTION OF THE PREFERRED EMBODIMENTS

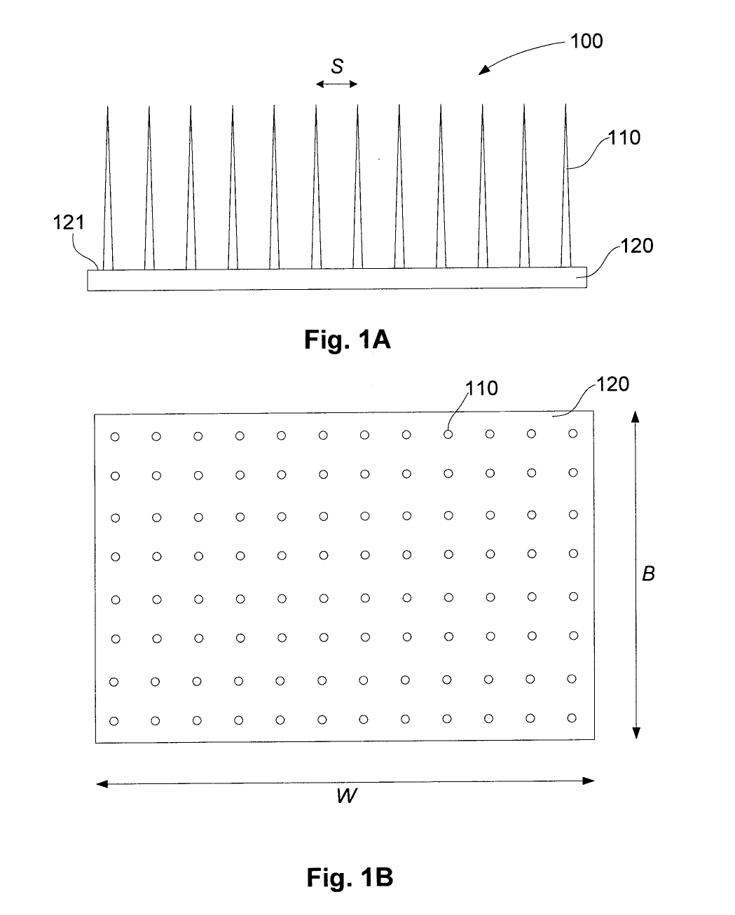

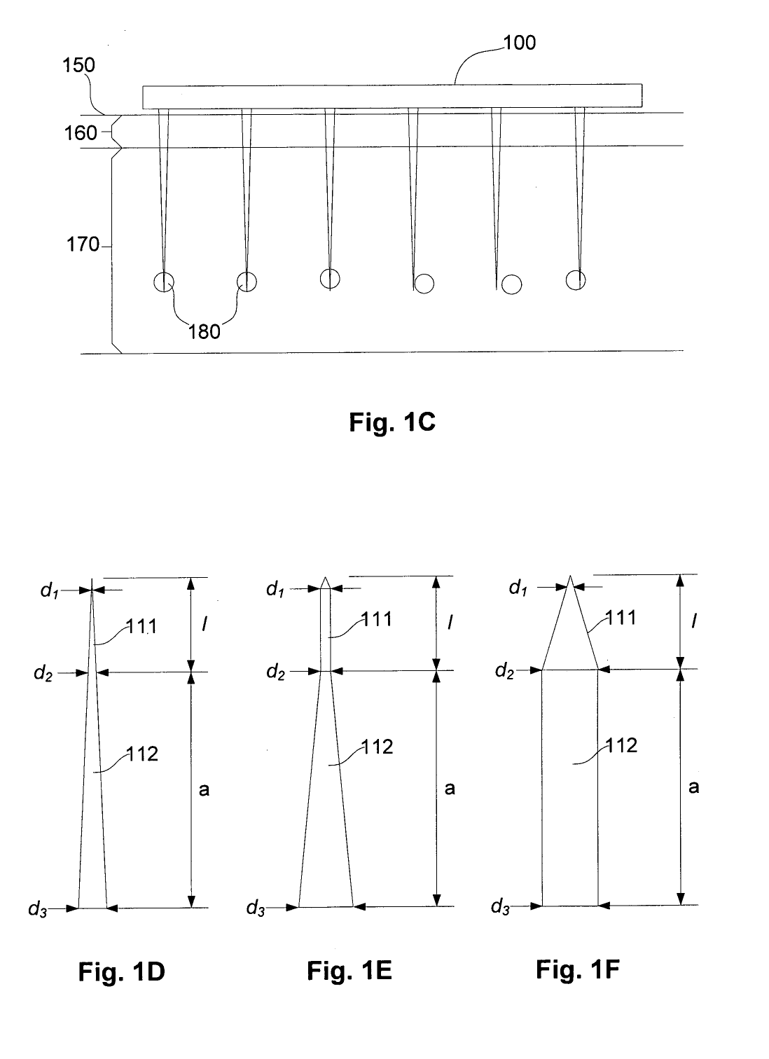

An example of a device for delivering material to targets within a body will now be described with reference to FIGS. 1A to 1F.

In this example, the device is in the form of patch 100 having a number of projections 110 provided on a surface 121 of a substrate 120. The projections 110 and substrate 120 may be formed from any suitable material, but in one example, are formed from a silicon type material, allowing the device to be fabricated using processes such as vapour deposition, silicon etching, Deep Reactive Ion Etching (DRIE), or the like. The projections are therefore typically solid, non-porous and non-hollow, although this is not essential.

In the example shown, the patch has a width W and a breadth B with the projections 110 being separated by spacing S.

In use, the patch 100 is positioned against a surface of a subject, allowing the projections to enter the surface and provide material to targets therein. An example of this is shown in FIG. 1C.

In this example, the patch 100 is urged against a subject's skin shown generally at 150, so that the projections 110 pierce the Stratum Corneum 160, and enter the Viable Epidermis 170 to reach targets of interest, shown generally at 180. However, this is not essential and the patch can be used to deliver material to any part or region in the subject.

It will be appreciated that the projections can have a variety of shapes, and examples of suitable projection shapes are shown in more detail in FIGS. 1D, 1E and 1F.

In one example, the projection includes a targeting section 111, intended to deliver the material or stimulus to targets within the body, and a support section 112 for supporting the targeting section 111. However, this is not essential, and a single element may be used.

In the example of FIG. 1D, the projection is formed from a conically shaped member, which tapers gradually along its entire length. In this example, the targeting section 111 is therefore defined to be the part of the projection having a diameter of less than d₂.

In FIGS. 1E and 1F, the structure of the projection may vary along its length to provide a defined targeting section 111 with a designed structure. In the example of FIG. 1E, the targeting section 111 is in the form of a substantially cylindrical shape, such that the diameter d₁ is approximately equal to the diameter d₂, with a tapered support section, such that the diameter d₂ is smaller than the diameter d₃. In contrast, in the example of FIG. 1F, the targeting section 111 is in the form of taper such that the diameter d₁ is smaller than the diameter d₂, with a cylindrical support section, such that the diameter d₂ is substantially equal to the diameter d₃.

In general, the support section 112 has a length α, whilst the targeting section 111 has a length l. The diameter of the tip is indicated by d₁, whilst the diameter of the support section base is given by d₃.

In use, the device can be used to deliver material to specific targets within the body or more generally to the blood supply, or tissue within the body and the configuration of the device will tend to depend on its intended use.

Thus, for example, if the patch is configured so as to ensure material is delivered to specific targets such as cells, then it may be necessary to select a more specific arrangement of projections than if delivery is provided more generally to the blood. To achieve this, the device can be provided with a particular configuration of patch parameters to ensure specific targeting. The patch parameters can include the number of projections N, the spacing S between projections, and the projection size and shape. This is described in more detail in co-pending application U.S. Ser. No. 11/496053.

In one specific example, a patch having a surface area of approximately 0.16 cm² has projections provided at a density of between 1,000-30,000 projections/cm². However, alternative dimensions can be used. For example, a patch for an animal such as a mouse may have a surface area of 0.32 to 0.48 cm², whereas as a patch for a human may have a surface area of approximately 1 cm². A variety of surface areas can be achieved by mounting a suitable number and arrangement of patches on a common substrate.

The projections typically have a length of between 10 to 200 μm and typically 90 μm with a radius of curvature of greater than 1 μm and more typically greater than 5 μm. However, it will be appreciated that other dimensions may be used.

If distinct targeting section and support sections are provided, the targeting section typically has a diameter d₂ of less than 20 μm, whilst d₁ is typically less than 5 μm and more typically less than 0.5 μm. The length of the support section typically varies depending on the location of the target within the subject. Example lengths include less than 200 μm for epidermal delivery and less than 1000 μm for dermal delivery.

In one example, at least part of the tips of the projections are coated. The coating process typically includes selecting a coating solution viscosity and then immersing at least part of tips of projections in a coating solution having the selected coating solution viscosity. The viscosity of the coating solution is selected to reduce the degree of capillary action between the patch and the coating solution during the immersion process, so such that substantially only tips of the projections are coated.

The selected viscosity will typically vary in accordance with patch properties such as the projection size, length, spacing, tip curvature, or the like, as well as the presence of any coating. These properties will typically affect the degree to which the patch exhibits hydrophilic behaviour, in which a contact angle between a liquid and the patch is less than 90°, or hydrophobic behaviour in which the contact angle is between 90° and 180°.

Whilst hydrophilic behaviour can be desirable as it reduces the extent to which the coating solution is repelled from the projections 110, thereby assisting the coating process, this also increases the effect of capillary action drawing coating solution along the projections 110 and onto the base 120, which can be undesirable. Even in the event that the patch is hydrophobic, in which case liquid is generally repelled from the patch, coating can still be achieved by suitable immersion of the projections, and even in this situation, capillary action can still occur.

However, by selecting a suitable viscosity, such as 1 Pa.S or above, this can reduce the effect of capillary action that can cause coating solution flow into contact with other parts of the patch, when the tips of the projections are immersed.

Accordingly, selection of a suitable viscosity can reduce the degree to which other parts of the patch, such as non-tip portions of the projections, or the patch substrate surface, are coated. As the other parts do not contribute to exposure of a subject to the coating, this can reduce the amount of coating applied to the patch, whilst still ensuring sufficient exposure of the subject. For the purpose of the following description, the term high viscosity refers to a viscosity of 1 Pa.S, or above, and optionally to a viscosity of 10 Pa.S or 50 Pa.S, whilst the term low viscosity refers to a viscosity less than 1 Pa.S and typically much less than 1 Pa.S.

For example, the coating can include a material to allow delivery of the material to the subject. In this instance, by applying at least the majority of the coating to the tips of the projections, this reduces the amount of coating, and hence the amount of material, required to deliver a desired amount of material to the subject. This in turn ensures that only a minimum amount of material is used in eliciting a desired biological response from a subject.

Reducing the amount of material required to elicit a response is particularly useful as this reduces the entire cost of a coated patch, thereby reducing the cost of treatments, or the like. This is also particularly useful when supply of the material is restricted for example due to limited availability, as can occur during mass vaccination programs, or the like.

The viscosity of the coating solution can be varied in any suitable manner, such as by adding a viscosity enhancer. For example, the viscosity enhancer can form between 0% and 90% of the coating solution. A range of different viscosity enhancers can be used and examples include honey, pectin, methylcellulose, sodium alginate, carboxymethylcellulose (CMC), gelatin, agar, and agarose and any other viscosity agents.

In addition to the above, appropriate selection of coating properties, such patch properties and immersion time, solution properties, such as surface tension, can be used to further control the coating process.

In one example, the coating solution is dried using a suitable technique, such as a gas flow, exposure to vacuum, or through control of the temperature and/or humidity of the environment in which the coating solution is dried.

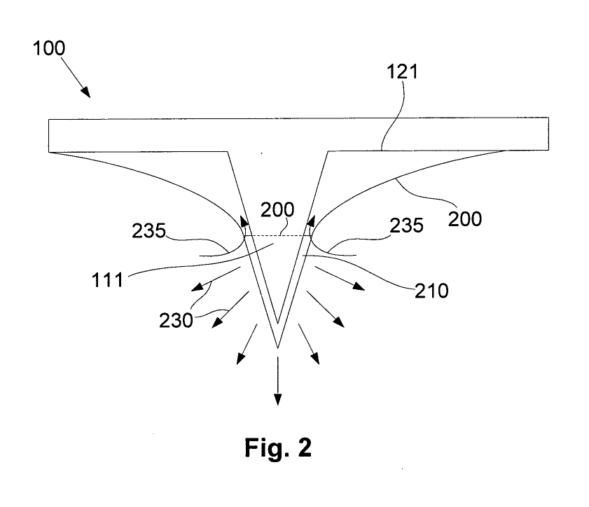

An example of the delivery of material to a subject will now be described with reference to FIG. 2.

In this example, the patch 100 includes coating 210 provided on the projection tip 111. Initially, when the patch 100 is applied to a subject, the projection tips 111 extend through the skin 200. The skin typically deforms in a region immediately surrounding the projection, with the skin bowing down away from the patch surface 121.

Upon insertion into the skin 200, coating 210 on the projection tips 111 below the skin surface 200, will immediately begin to hydrate and dissolve, thereby being dispersed into the subject, as shown by the arrows 230. This allows the coating to be released into the skin within seconds after insertion.

The projection tips can be coated using any suitable technique, such as immersion, dipping or the like. In one example, the depth of immersion can be used to control a length of the coating on the projections. In one particular example, coating is performed using apparatus that includes a positioning system that allows exposure of the projection tips to coating solution to be controlled, thereby controlling the immersion depth and hence the coating length. For example, the patch can be positioned in a well containing coating solution, so that only the projections of the tips are immersed. Such positional control can be achieved using an electronic micro-positioning system, abutment between the projection tip or base, or any other suitable mechanism.

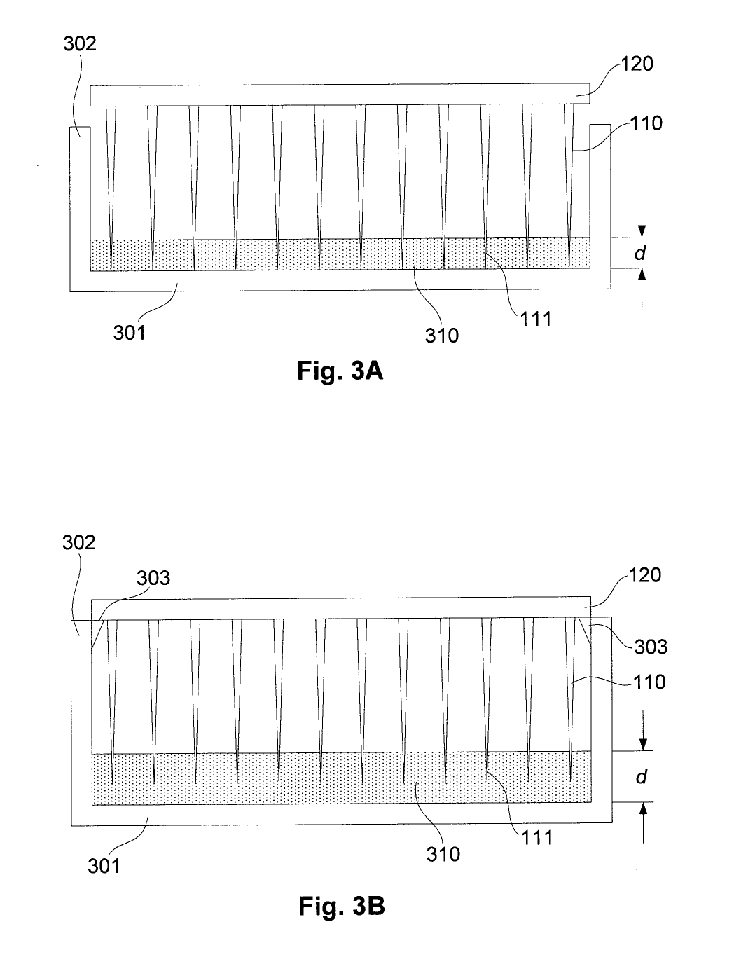

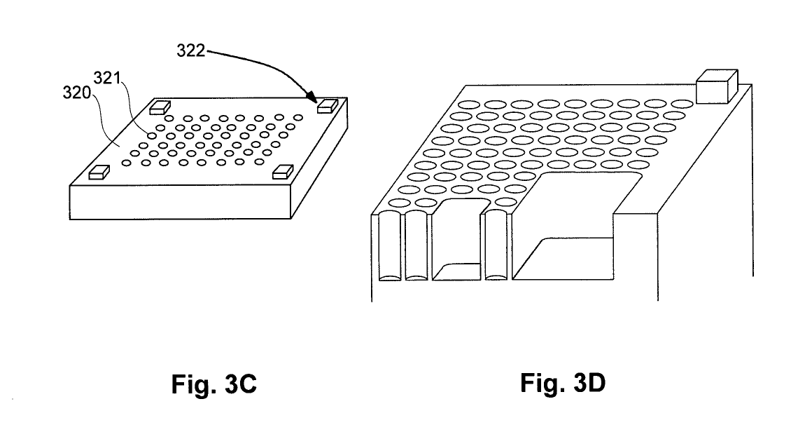

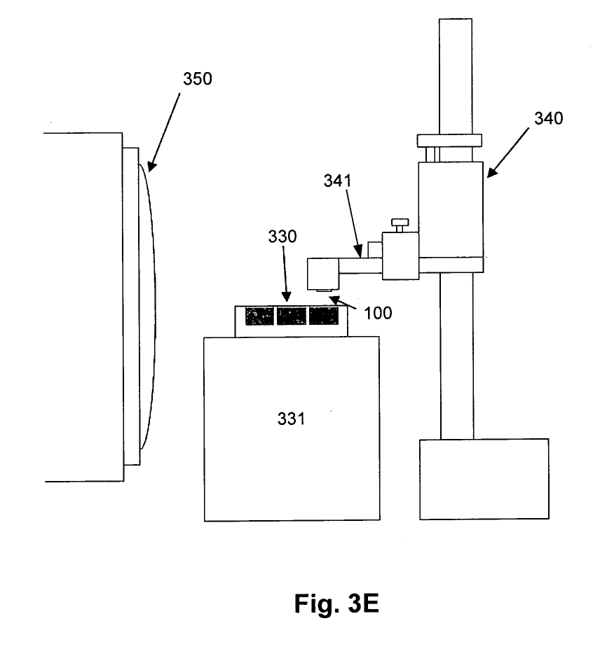

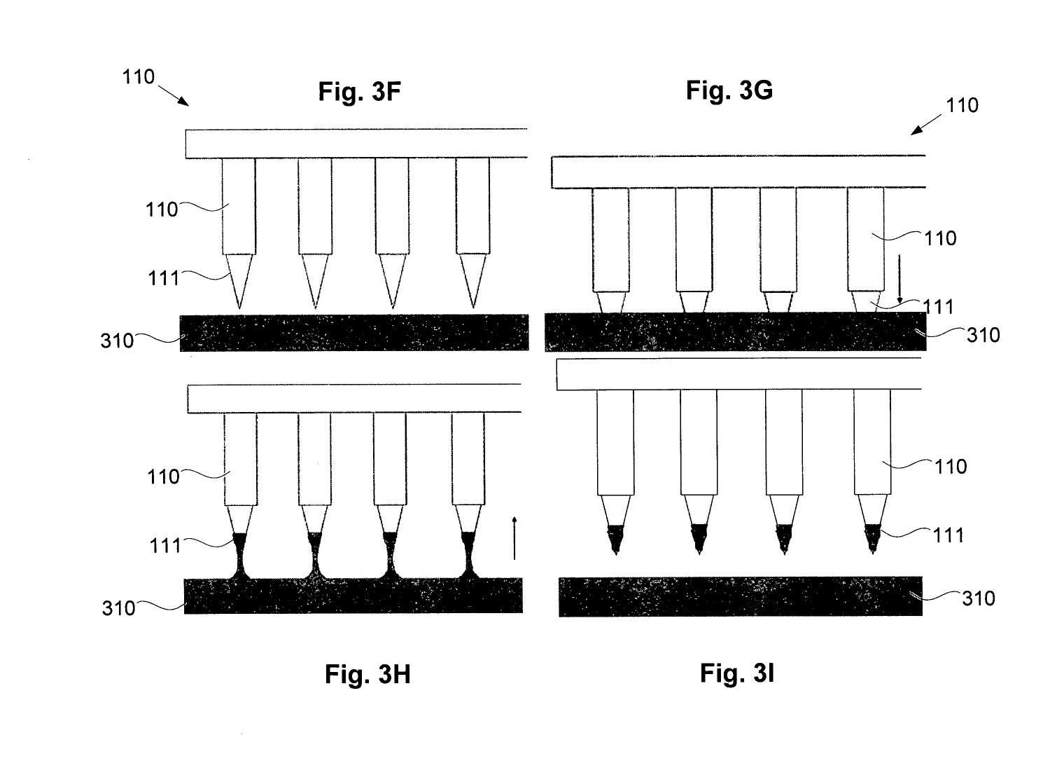

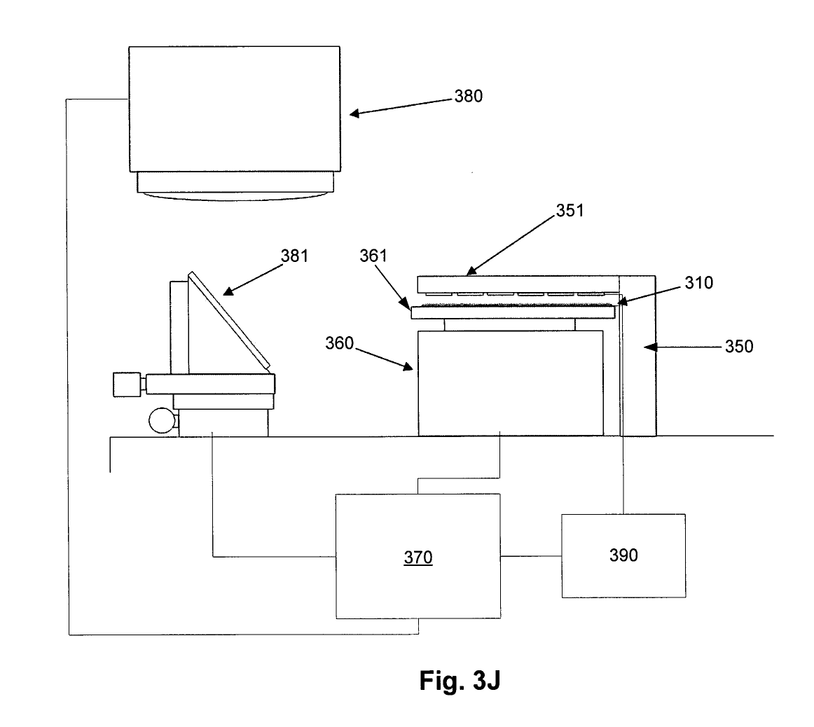

Examples of suitable apparatus will now be described with reference to FIGS. 3A to 3F.

In the example of FIG. 3A, the apparatus includes a well 300, having a floor 301 and walls 302, having the coating solution 310 provided therein. The depth d of the coating solution is controlled so that upon insertion of the patch into the well 300, the projections 110 abut against the floor 301 so that only the tips 111 of the projections 110 are immersed in the coating solution 310. As a result, only the tips 111 of the projections are exposed to the coating solution, with the relatively high viscosity of the coating solution preventing capillary action from allowing the coating solution to coat the remainder of the projection.

In one example, the well can be made of a soft polymer, thereby allowing the well to absorb any force applied by the projections, thereby reducing the chance of projections breaking. The patches can also be inserted by a force sensitive device, such as an electronic controller, which is adapted to stop movement of the patch if it detects that the projections are in contact with the well, for example if the applied force used to move the patch rises over a set value such as 10 mN.

This example is useful as it avoids the need for complex alignment and micro positioning system, thereby rendering the coating process cheaper than if such devices are used. This example is particularly suited for patches having long projections, such as 500 μm.

In the example of FIG. 3B, the apparatus the walls 302 include stops 303. The depth d of the coating solution is again controlled so that upon insertion of the patch into the well 300, the substrate 120 abuts against the stops 303 so that again only the tips 111 of the projections 110 are immersed in the coating solution 310. In this example, this is achieved without requiring contact of the projection tips 111 with the floor 301 of the well 300, which can cause damage to projection tips 111, depending on the nature of the projections.

The use of deep wells also makes filling the wells with coating solution easier as it is generally easier to fill in a large volume of coating solution. Additionally, as coating solution in the well becomes depleted, the coating depth can be controlled by adjusting the size or the position of the stops 303. The well can also be significantly larger than patches in area, which gives even more tolerance when inserting the patch into the well.

In both of the above examples, a single well can be used to coat all of the projections on the patch. However, in alternative examples, multiple wells can be provided, each of which contains coating solution. An example of this is shown in FIGS. 3C and 3D, which show a mould 320 including a number of wells 321. Stops 322 are provided to allow the depth of penetration of the projections 110 into the wells 321 to be controlled.

In one example, the patch can include areas having respective projections which are adapted to be inserted into respective ones of the wells 321. This allows respective areas of the patch to be coated with different coating solutions, allowing for example, delivery of different biological agents to a subject.

In the example of FIG. 3E, the apparatus includes a well 330 containing coating solution, which is typically supported on a support, such as a table, bench, platform, or the like. A positioning system 340 is provided, which in one example is a Vernier height scale, having a support arm 341 extending outwardly therefrom. In use, a patch 100 is mounted on an underside of the support arm 341, allowing the patch to be positioned above the well 330, with the projections 110 extending towards the well 330, as shown in FIG. 3F. A microscope, or other imaging system 340, is provided, allowing the separation between the projections and the well 330 to be observed.

In this example, manual control of the Vernier scale is used to lower the projection tips 111 into the well 330, with the process being observed to ensure that the tips 111 are at least partially immersed in the coating solution 310, as shown in FIG. 3G. After an immersion time, such as a few seconds, the Vernier scale is used to move the patch 100 away from the well.

On slow removal of the projection tips 111 from the coating solution 310, a thin film is left on the projection tips 111, as shown in FIGS. 3H and 3I.

It will be appreciated however that a manual positioning process can be slow and inaccurate. Accordingly, further improvements can be achieved using controlled micro-positioning apparatus, an example of which will now be described with reference to FIG. 3J.

In this example, the apparatus includes a support 350, having an arm 351. The arm 351 extends laterally from the support 350, above a positioning system 360. The position stage includes a 6-axis controlled platform 361, whose position is controlled using a suitable controller 370. The platform 361 typically has a layer of coating solution provided thereon, as shown at 310. In one example the coating solution is confined using a well, although alternatively this can remain in position solely due to the high viscosity.

An imaging system 380 is provided, with an adjustable mirror 381 being used to allow the relative separation of the arm 351 and the stage to be viewed. In one example, the controller is coupled to the mirror 381 and the imaging system 380, allowing the position of the platform 361 to be controlled by the controller 370.

In this example, one or more patches may be mounted to an underside of the arm 351, such that the projections 110 are facing the coating solution 310. The controller 370 will then raise the platform 361 towards the arm 351, thereby immersing the projection tips 111 in the coating solution. During this process, the controller 370 can use image processing software to determine the relative separation of the projections 110 and the coating solution, thereby allowing accurate immersion to be achieved. Following immersion for a predetermined time, the controller 370 lowers the platform 361, removing the projection tips 111 from the coating solution.

It will be appreciated that the controller 370 is typically in the form of a processing system, such as a suitably programmed computer system, a custom hardware controller, or combination thereof.

Additionally, any suitable form of system for controlling the relative position of the platform 361 and the arm 351 may be used. For example, the platform 351 could include stops, which abut against the arm 351. The controller could be configured to detect the force required to move the platform 361, with an increase in force indicating contact has occurred between the stops and the arm 351, allowing the controller 370 to halt movement. In this instance, the imaging system 380 may not be required.

Alternative methods for determining the separation include the use of optical or magnetic sensors provided on either the arm 351 or the platform 361, as will be appreciated by person skilled in the art.

Further alternatives include sensing using conductivity or resistance measurements made between the projections 110 and the coating solution 310. When the projections are not in contact with the coating solution the conductivity will be low and resistance high. However, as the projections penetrate the coating, the conductivity will increase and resistance will decrease. Finally when the coated projections are removed from the coating solution, the conductance will be low and resistance high. These electrical properties are used to automate the dipping process, making mass production more reliable and controllable.

Accordingly, in this example, the controller 370 could be coupled to a signal generator 390, which is capable of applying an electrical signal to the projections 110 and the coating solution 310, and communicating information relating to the signal to the controller 370. Thus, for example, the signal generator could apply a predetermined potential difference between the projections and the coating solution, with an indication of the resulting current being used by the controller 370 to establish whether the projection tips are immersed, and optionally the degree of immersion.

A further benefit of this arrangement is that it can be used to assist coating of the projections through electrophoresis. Adjusting the pH of the coating solution imparts a charge on payload molecules, allowing an electrical current to be used to complete a circuit between the projections and the coating solution. This method, called electrophoresis, concentrates the entire payload in the coating solution onto the projection tips, thus eliminating payload wastage. This process also results in highly controllable and reproducible payload coating.

Accordingly, in one example, the apparatus applies a current through the projections and the coating solution both for the purpose of sensing the degree of immersion, as well as to assist in the coating process through electrophoresis.

It will be appreciated that using an automated process similar to that described above allows one or more patches to be controllably and reliably immersed, thereby increasing the rate at which patches can be coated.

Using computer control also assists to provide additional control over the coating. For example, the controller 370 can be adapted to insert or remove the projection tips in accordance with a predetermined velocity profile. Slow immersion could be used for example to ensure even exposure of the entire projection tip, whilst rapid removal may be used to prevent excess coating solution remaining attached due to the high viscosity of the material.

Additionally, following insertion, the projection tips can be agitated within the coating solution, for example by reciprocal oscillation of the patch relative to the coating solution, or vice, versa. Oscillating the patch and coating solution in this fashion help further expose the projection tips to the coating solution, and help ensure an even coating results. In one example, the oscillation amplitude and frequency during immersion can therefore be controlled, to ensure complete exposure of the projection tips to the coating solution. Typically the amplitude of the oscillation is in the range of 0.01 to 100 μm, whilst the frequency of the oscillation is in the range of 1 to 10,000 Hz. In one example, the amplitude of oscillation is about ±1 μm and the frequency is about 400 Hz. Oscillation of the patch can also be performed post-immersion to remove excess coating solution.

Agitation of the coating solution can also be used to ensure the coating solution surface is flat prior to immersion of the projections. This helps allow each of the projections to be inserted an equal depth into the coating solution. Agitation during application of the coating can also assist in ensuring contact between the projection surface and the coating material, thereby helping the even coating of the material on the projection tips.

It will be appreciated that these techniques are for the purpose of example only, and other techniques may be used for applying coating solution to only the tips of the projections.

Thus, the above described apparatus can assist in allowing projection tips to be immersed thereby controllably coating the projection tips. The technique avoids the need for a physical mask to avoid capillary action, or complex equipment to align the projections with the mask.

Accordingly, the above described examples allow a very viscous coating solution or gel to be used to coat projection tips. The high viscosity avoids capillary action that can lead to contamination of the base of patches and the lower regions of the projections with coating. As these parts are not inserted into skin, the majority coating on these parts of the patch will not be effective for the rapid delivery of material to a subject, which can therefore result in unnecessary use of coating solution, and hence any contained material.

When performing the coating process it is typical to select coating properties, which can include patch properties, such as projection size and spacing, coating solution properties, such as surface tension and an immersion time.

The coating process can also be influenced by other coating properties such as the velocity profile for the insertion and removal of the projection tips from the coating solution, as well as the nature of any agitation, such as the amplitude and frequency of an oscillation.

Appropriate selection of the coating properties can be used to control an amount of coating on the tips. This can also further assist in ensuring that only tips of the projections to be coated, as will be described in more detail below. As patch properties may impact on the coating process it is typical to first determine patch properties and then use this information to allow appropriate other properties to be selected.

In general the coating solution includes at least a material such as a therapeutic agent and examples of suitable materials include:
nanoparticles;
a nucleic acid or protein;
an antigen, allergen, with or without, adjuvant, or adjuvant alone;
parasites, bacteria, viruses, or virus-like particles;
quantum dots, SERS tags, raman tags or other nanobiosensors;
metals or metallic compounds; and,
molecules, elements or compounds.

Examples of preferred formulations include a solution containing DNA having a concentration of between 0.01 mg/ml and 10 mg/ml or protein having a concentration of between 0.01 and 100 mg/ml.

The agent or other material may be dissolved in a suitable solvent or held in suspension in a suitable carrier fluid, as will be appreciated by those skilled in the art. In one example, the solvent is water, although alternatively other suitable solvents can be used.

The solution properties are typically controlled through the addition of one or more other agents such as a detergent or other surfactant, and an adjuvant. These ingredients can be provided in a range of different concentrations. A range of different surfactants can be used to modify the surface tension of the coating solution, such as any detergent or any suitable agent that decreases surface tension, and that is biocompatible at a low concentration.

In one example, the coating solution or gel has a viscosity of over 10 Pa.S, when measured at a temperature of 25° C. and a shear strain rate of 100 sec⁻¹. The thickness of the coated vaccines is typically less than 10 μm but may be greater depending on the intended use and the nature of the vaccine. There are three major components of the coating solution the viscous agent, a detergent, and the vaccine. The viscous agent can be methylcellulose, carboxymethylcellulose, gelatin, agar, agarose, honey, pectin, sodium alginate or any biocompatible polymer. The detergent decreases surface tension and can be composed of poloxomer 188, triton-X 100, NP40, or any detergent that is biocompatible at low concentrations. The vaccine can be composed of DNA or protein and can also contain adjuvant. The concentration, viscosity and surface tension will have influence on the film thickness, morphology and payload of coating.

It will be noted that whilst any viscous agent can be used, honey, pectin and sodium alginate are particularly useful as they are all approved for human use.

Accordingly, the above described examples provide method for coating therapeutic agents including vaccines on to projection tips on a patch, to thereby allow for their rapid release when the patch is applied to a subject. The method provides substantially uniform and controllable coating of therapeutic agents like DNA or protein vaccine onto the patches. The method can be applied to any form of patch.

Further variations and options will now be described.

For example, the patch and/or projections can be coated with a thin layer of a suitable material, prior to application of the coating solution. This can be used to modify the surface properties of the patch, for example to make the surface more or less hydrophilic. This assists in ensuring that at least some of the coating solution adheres to the projection tips. The hydrophilic nature of the patch can be achieved by coating the patch with a suitable material, prior to immersing the tips.

Thus, in one example, the patch is coated with a layer of methylcellulose using any suitable coating technique. In one example, this is achieved using the above described technique, with at least tips of the projections being immersed in a coating solution. Alternatively this can be achieved using a gas coating technique, described for example in copending patent application number WO2009/079712. In this instance, a coating solution containing methylcellulose is applied to at least the projections, with a gas jet being used to distribute and dry the coating solution. These techniques can be used to provide a hydrophilic coating on at least the projection tips, thereby assisting in ensuring coverage of the projection tips. In another example, 3-APTES is reacted with the siliceous surface of a silicon patch, to thereby create an aminopropyl substituent on the surface of the projections, which in turn results in a hydrophilic behaviour.

In one example, it will be appreciated that the degree to which the patch is hydrophilic may also depend on the patch configuration and in particular, on patch parameters such as the projection size and shape and the projection spacing S. Accordingly, in one example, the contact angle, which determines whether a patch is hydrophilic or hydrophobic, is also used in determining the desired viscosity for the coating solution.

As described above, the coating solution is typically selected to have a suitable viscosity, which may be achieved through the use of viscosity enhancers. Similarly, surfactants can be used to control the surface tension.

The surfactant can be a detergent or any suitable agent such as poloxomer 188, triton-X 100, NP40, Quil-A, or any detergent that is biocompatible at a low concentration. The concentration of the detergent is from about wt. 0% to about 90% of the coating solution, depending on the required solution properties.

A vaccine adjuvant may also be added to the coating solution for enhancing immune response to vaccines. In one example, the adjuvants used include Quillaja saponins, such as Quil A, QS 21, QS7 or other purified saponin adjuvants. Use of Quil-A and other similar saponin adjuvants can be particularly beneficial as Quil-A not only acts as a surfactant for coating purposes but also as the vaccine adjuvant. Furthermore, due to Quil-A effectiveness in reducing the surface tension of the coating solution, this can in turn help in reducing the amount of excipients used for coating.

Other amphipathic immunostimulatory compounds such as dimethyldioctadecylammonium bromide or chemically modified immunostimulatory molecules to give detergent properties can also be employed.

The viscosity agent can be selected from honey, pectin, methylcellulose, carboxymethylcellulose, sodium alginate, gelatin, agar, agarose, pectin, or any other viscosity agent, which can be any substance that modifies the viscosity of the coating solution. The concentration of the viscosity agent is typically from about wt. 0% to about 90% of the coating solution.

Whilst a range of therapeutic agents can be used, in one example the agents are vaccines. The vaccine can be composed of any suitable material and may include DNA, protein, viral (attenuated or split), VLPs, or the like, as described in more detail below. Additionally an adjuvant may also be included. The concentration of DNA in the coating solution can be from 0.01 mg/ml to 10 mg/ml. The concentration of protein in the coating solution can be from 0.01 to 100 mg/ml.

The material can include nanoparticles to provide a nanodelivery system. For example the coating can include DNA containing nanoparticles.

In one example, the nanoparticles are multilayered nanoparticles. Outermost layers of the nanoparticles can include cell targeting and cell-entry facilitating molecules. The next layer can include intracellular targeting molecules for precise delivery of the nanoparticle complex inside the cell of interest.

Molecular biosensors can be used to confirm the presence of expected molecules as a surrogate molecule for signs of infection, for activation in radiation damage, or other criteria, prior to delivery of counter-measure molecules such as vaccines, drugs, or gene therapy. The biosensors can also be used as a feedback control mechanism to control the proper amount of vaccine/drug/gene delivery for each cell.

Further, the nanodelivery system can be used to restrict any cells from encountering the drug unless that cell is specifically targeted. Successful targeting can be verified by 3D multispectral confocal microscopy. These single cell molecular morphology measurements can be extended from individual cells, to other cells in a tissue in tissue monolayers or tissue sections.

This example can be used to provide a nanomedical system and method that can be used for diagnostics, therapeutics, vaccines, or a combination thereof by use of a multilayered nanoparticle system. The multilayered nanoparticle system can be built on a nanoparticle core of bio-polymer, polystyrene, silica, gold, iron, or other material.

The concentration, viscosity and surface tension will all influence the thickness, morphology and payload of coating. In the most examples, the thickness of the coated vaccines can be from 10 nm to 10 μm, although greater thicknesses may be used depending on the material being delivered to the subject, and the circumstances in which patch is to be used.

The amount of resulting dry coating on the projections can be controlled by the concentrations of excipients in coating solution, as well as the surface area of the projections, although as mentioned above, selection of an appropriate surfactant, such as Quil-A can avoid the need for unnecessary excipients.

In one example, a payload, such as material in the coating solution, can be concentrated on the projections through an appropriate technique. This can be achieved in any suitable manner that assists attraction of the payload, such as through the use of a suitable coating applied to the projections, or through the use of active techniques, such as electrophoresis.

When providing a coating for attracting the payload, the coating may be applied in a manner similar to that described above, so that the coating is provided on the projection tips only. In this instance, a second coating process is performed including the payload, with this being attracted to the projection tip only, through the attracting to the underlying coating provided on the projection tips only.

In the case of electrophoresis, adjusting the pH of the coating solution imparts a charge on payload molecules. Electrical current can be used to complete a circuit between the projections and the coating solution. This concentrates the entire payload in the coating solution onto the projection tips.

It will be appreciated that the above described techniques further assist in ensuring coating of payload is confined to the projection tips, thereby reducing or eliminating payload wastage.

In one example, only the projection tips are coated. Consequently, when the patch is placed on the skin, substantially all of the coated therapeutic agent can be rapidly delivered into the skin from the projections. This is useful where it is desired to provide rapid delivery of an agent.

In one example, the projections can be coated a single time. In a further example, the projection tips can be coated a number of times. This can be used to allow a required thickness of coating to be achieved. In addition to this however, this allows different coating regimes to be used, which in turn allows greater control over the coating process. Additionally, this can be used to provide protective coating layers to prevent material being inadvertently delivered during penetration of the projections into the subject, as well as to control the region to which material is delivered within the subject. Multiple coating layers can also be used allow different materials to be provided to different regions of a subject, or to allow different materials to be delivered to a subject in a sequential manner.

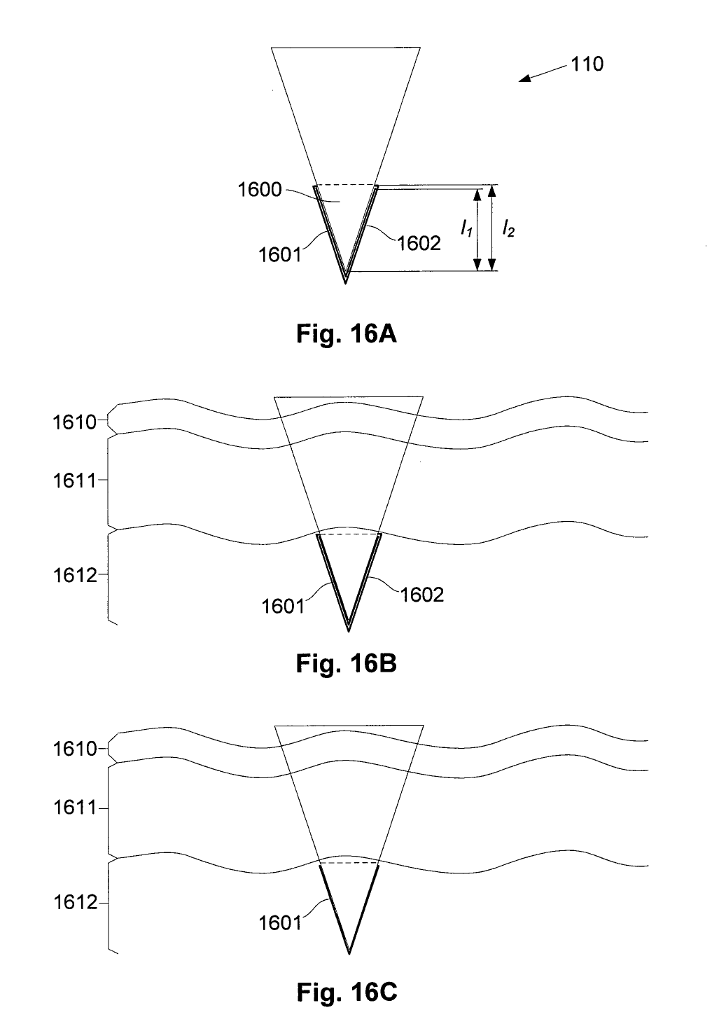

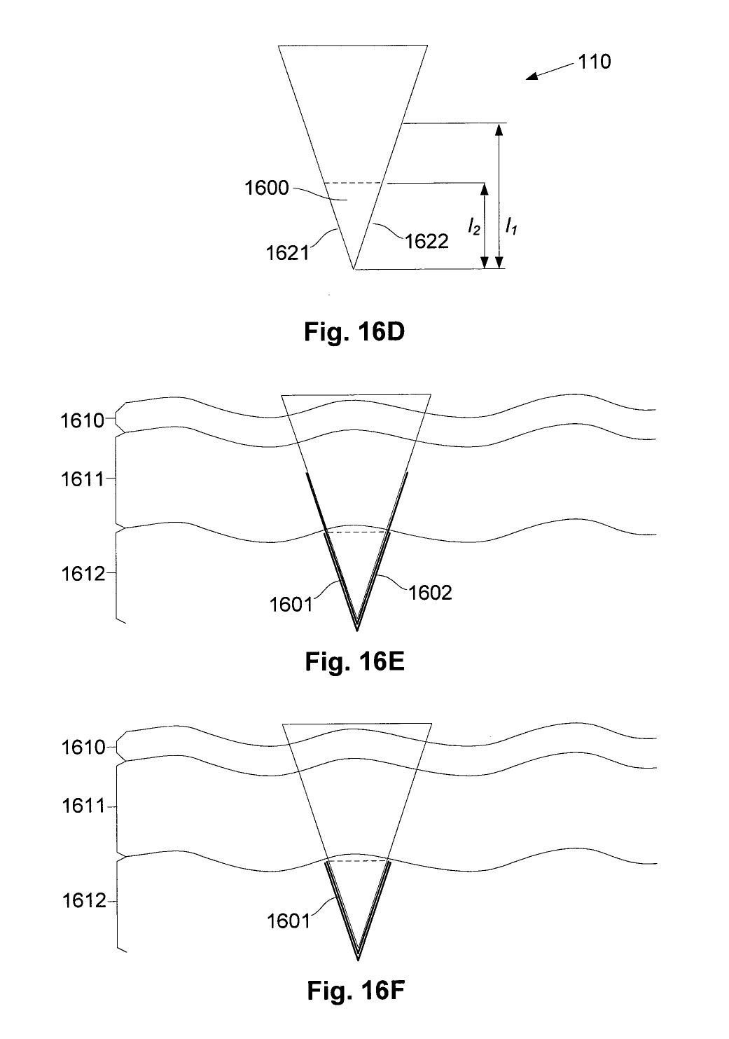

An example of a projection tip having multiple coatings will now be described with reference to FIG. 16A. In this example, the projection 110 includes a first layer 1601 of a first coating material, and a second layer 1602 of a second coating material, provided on a projection tip 1600. To achieve this, the projection would first be coated with the first coating material to thereby form the first layer 1601, which is then allowed to dry, before the second coating material is applied to form the second layer 1602.

In this instance, the coatings may be applied using any suitable technique, such as the gas jet coating technique described for example in copending patent application number WO2009/079712. However, typically at least one of the layers is coated by immersing the projection tips in a coating solution having a viscosity selected so as to reduce capillary action, thereby allowing the length of the projection that is coated by the first and second coatings l₁, l₂, to be carefully controlled.

In this example, the length l₂ is slightly greater than the coating length l₁ so that the first coating layer 1601 is completely encompassed by the second coating layer 1602. This can be used to allow the second layer 1602 to act as a protection layer, thereby preventing the first layer 1601 of material being dislodged or otherwise affected during penetration of the subject by the projection. Alternatively, the first and second layers 1601, 1602 can contain respective first and second active ingredients, so that in this example, the second active ingredient is delivered to the subject before the first active ingredient.

An example of penetration of the subject is shown in FIG. 16B. In this example, the projection 110 is urged against the surface 1610 of the Stratum Corneum 1611, so that the projection 110 penetrates the Stratum Corneum 1611 and the Viable Epidermis 1612, with the projection tip 1600 entering the dermis 1613.

When the projection initially enters the subject, the second layer protects the first layer so that as the projection penetrates the subject, any coating material that is sheared off during insertion will be the second coating layer. This ensures that the first layer 1601 can penetrate to the dermis without delivering the material therein to the Stratum Corneum 1611 or Viable Epidermis 1612. Once in position, as shown in FIG. 16B, the second layer 1602 dissolves, thereby exposing the first layer 1601, as shown in FIG. 16C, ensuring that material in the first layer 1601 is delivered to the Dermis 1612 only.

Thus, in this example, the second layer 1602 acts as a protective barrier to prevent material in the first layer 1601 being delivered to parts of the subject other than the intended target, in this case the Dermis 1612. It will be appreciated however that alternatively the different coating layers may contain different active materials, allowing different active materials to be delivered in sequence. It will also be appreciated that targeting of the Dermis 1612 is for the purpose of example only, and that alterative arrangements may be used to target different regions within the subject.

An example of this will now be described with reference to FIGS. 16D to 16F.

In this example, the length of the first coating 1621 is significantly greater than that of the second coating 1622. As a result, when the projection enters the subject as shown in FIG. 16E, the first coating layer 1621 is exposed only in the Viable Epidermis 1612. If the second coating layer is a protective layer that does not dissolve, then as shown in FIG. 16F, material can be controllably delivered to the Viable Epidermis 1612 only.

It will therefore be appreciated that by using multiple coating layers this can provide significantly enhanced controllability of delivery of material to a subject.

In one example, an overlying second coating layer can be used to protect an underlying first coating layer, so that the first coating layer containing active material is protected during insertion into the subject. This can prevent material in the first layer being delivered to unwanted regions of the subject, as may arise for example if the first layer were unprotected and shear forces created during insertion caused the first layer to be removed from the projection tip before the tip reaches the desired region within the subject.

In a further example, the second coating layer could be adapted to dissolve in a controlled manner to thereby provide for a timed release of material from the first layer.

It will also be appreciated that controlling the relative coating lengths of the different layers can also help control the regions of the subject to which material is delivered. Finally by providing different coating layers with different coating materials this can allow different materials to be delivered to different regions of the subject, or to be delivered in a sequential manner.

The projections can be coated with DNA or protein vaccines. However, in addition to this, many other reagents can be coated using this process including both inorganic and organic materials. Example coatings used include inorganic materials such as EtBr, or organic materials such Evans blue, Dextran, DiD, or the like.

Consequently, the resulting patch can provide small and densely packed projections that can be uniformly and controllably coated. This allows vaccines or other agents to be subsequently delivered to highly immunologically sensitive cells within the epidermis, dermis, or to the blood, muscle, or other tissue as required. Furthermore, by providing the coating on the tips, this maximizes efficient use of the coating material.

In use, the coated and dried projection patches are applied to the skin of a mammal by placing the patch on the skin, and/or through the use of an applicator to apply the patch to the skin with a predetermined force, velocity, strain rate, or the like. The coated and dried projection patches can be tested on skin or skin analogs and the conditions for optimal coating release determined. These conditions include patch geometry, application time, force, velocity, strain-rate of insertion, temperature, humidity, location, and skin pre-treatment. This process can be done in vitro, ex vivo or in vivo, and a number of experiments investigating the effectiveness of the above described coating methods will now be described.

It will be appreciated that the final release of the therapeutic agent can also be influenced by several of the coating properties such as the inclusion of excipients, as well as the coating thickness, and testing again allows optimum coating properties such as those outlined above, to be determined.

The in vitro method involves dipping coated patches in a solvent that can dissolve the coating material. The nature of the solvent will depend on the coating provided, but will typically include water, tris buffered saline (TBS), phosphate buffered saline (PBS), or the like.

The ex vivo release assay can be used to assess release from the coated and dried projection patches, using donor excised skin. A patch of skin is dissected from a donor (i.e. mouse, pig, rat, human) and kept at −20° C. for less than 7 days prior to use. The skin is warmed to 37° C. and the patches coated as outlined above are applied under a variety of conditions. The patches can be coated with fluorescent dyes such as FITC, Evans Blue, Propidium Iodide, Ethidium Bromide, Alexa Flur dyes. The patches can also be coated with DNA or proteins that are labelled with fluorescent dyes. Alternately, the patches can be coated with fluorescent dye labelled polymers like dextran, agarose, agar or any other biocompatible polymer that approximates the size, shape, and chemical nature of DNA and protein vaccines.

The release of these fluorescently labelled agents in skin can be monitored by methods including multi-photon/confocal microscopy, fluorescence microscopy, spectrofluorometer, and flow cytometry. Multi-Photon/Confocal microscopy can give real time, 3D patch release information that is necessary for optimizing the device coating and application.

In in vivo release testing, a coated projection patch is applied to the skin. After the application, analysis was carried out as discussed for the ex vivo testing protocol. Alternately, a portion of the skin treated with the projection patch is excised. The outer layer of the skin is peeled and trimmed as required. The skin is snap frozen in liquid nitrogen and then pulverized to a fine powder.

For DNA vaccine delivery, the DNA is extracted with a Qiagen extraction kit and a standard curve employed to determine the amount of DNA with semi-quantitative Polymerase chain reaction (PCR).

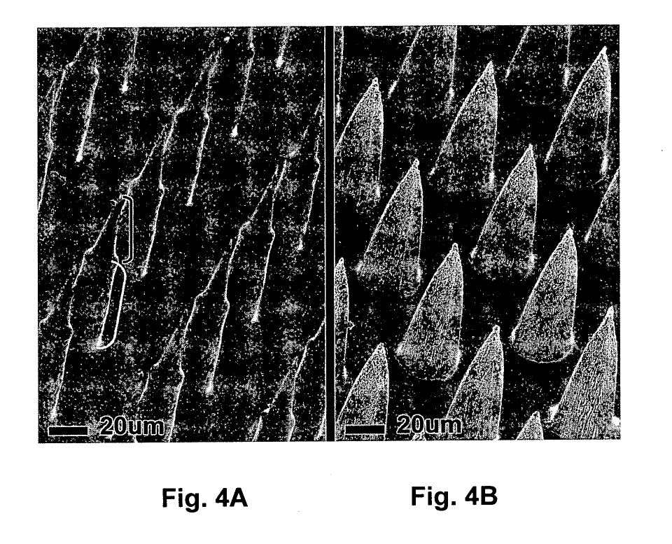

Specific examples of patches used for the experiments are shown in FIGS. 4A and 4B. The patches were fabricated from silicon using a process of Deep Reactive Ion Etching described for example in copending application number WO2009/097660. The projections are solid silicon. Some of the patches were sputter coated with a thin layer of gold (400-1500 nm in thickness). The morphology of MNP patches and coating were characterized by a JEOL scanning electron microscope 6400 or Philips XL30.

The projections shown in FIGS. 4A and 4B have lengths of 120 and 100 μm, and diameters of 28 and 35 μm, respectively. The spacing between the centres of adjacent projections is about 70 μm for both configurations. An individual patch is 5×5 mm in size and the central 4×4 mm area contains 3364 densely packed projections. As shown in these examples, the projections can have different geometries, with the projections of FIG. 4A having a stepped geometry created through a two stage etching process, described for example in copending application number WO2009/097660, whilst the projections of FIG. 4B have a sloped configuration.

Specific examples of coated patches will now be described. In this example, the patches are prepared using the following protocol:

1. Patches are cleaned in glycerol:H2O (1:1) for 10 minutes and then flushed with plenty of water;

2. Cleaned patches are dried with nitrogen gas flow;

3. Coating solution is made of pectin, sucrose, Quil-A and vaccine or a vaccine surrogate such as rhodamine-dextran. The concentrations of chemicals are adjusted to suit different requirements and examples are provided below;

4. Approximately 100 μl of the coating solution was placed on a planar substrate and spin for 3 seconds to form a thin layer with very smooth surface;

5. The patch was controllably dipped into the coating solution for 10 seconds by a micropositioning system monitored by a stereo microscope as described above with respect to FIG. 3E;

6. The patch was removed from the coating solution and dried in air or by a nitrogen gas jet so that coating length was controlled by the dipping depth in coating solution.

Three types of coating solutions were used for coating MNPs.
Dissolved 2 mg/ml of methylcellulose, 2 mg/ml of Quil-A and active materials in water. The solution is referred to “methylcellulose solution” in this paper.
Dissolved 2.1 g/ml of sucrose in water and then heated to 80° C.; added 1 g/ml of pectin for dissolution; added active materials (e.g. fluorescent dyes, vaccines) when the solution cooled down to 35° C.; allowed the solution to set at 4° C. for 24 hours before it was used for coating experiments. The coating solution was applied to the projections, with both at room temperature. The solution is referred to “pectin solution” in this paper.
Dissolved 20 mg/ml of sodium alginate, 600 mg/ml of sucrose, and active materials (e.g. fluorescent dyes, vaccines) in water; put the solution at 4° C. for 24 hours to remove bubbles produced in the dissolution. The coating experiments were performed at room temperature. The solution is referred to as “sodium alginate solution” in this paper.

Delivery of coating to skin was investigated using the following:
1. Excise a 5×5 mm piece of skin.
2. Spread the skin flat on a No 1 cover slip.
3. Attach a microneedle device coated with fluorescent material on a sprung device (patch application device).
4. Apply the coated patch to the skin (22° C., 70% humidity, sprung device, 1.9 m/s, patch on skin for 1 minute).
5. Examine the release under a confocal/Multi-Photon microscope.

To evaluate the effectiveness of material delivery, the microneedle device can be coated with fluorescent dyes such as FITC, Evans Blue, Propidium Iodide, Ethidium Bromide, Alexa Fluor dyes. The device can also be coated with DNA or proteins that are labelled with fluorescent dyes. Alternately, the device can be coated with fluorescent dye labelled polymers like Dextran, agarose, agar, and any other biocompatible polymer that approximates the size, shape, and chemical nature of DNA and protein vaccines. The release of these fluorescently labelled agents in skin can be monitored by methods including confocal microscopy, fluorescence microscopy, spectrofluorometer, and flow cytometry. Confocal/Multi-Photon microscopy can give real time, 3D patch release information that is necessary for optimizing the device coating and application.

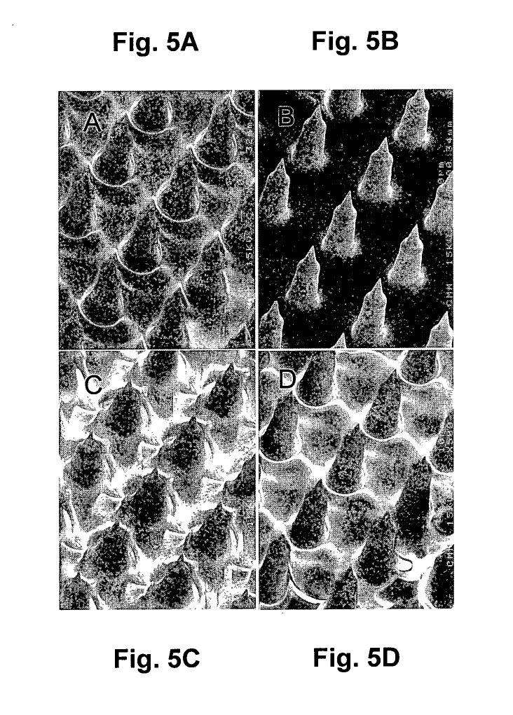

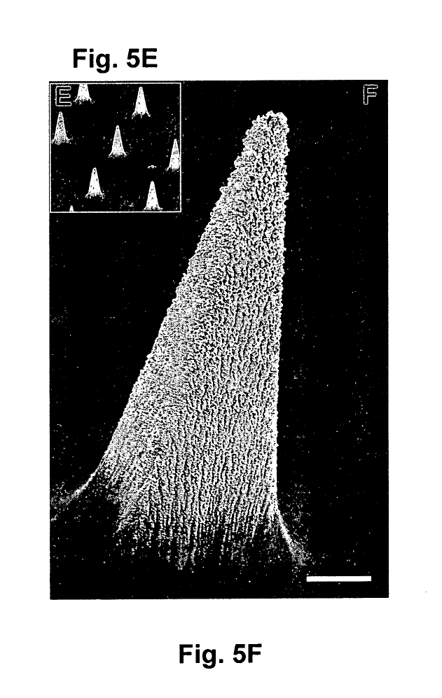

FIGS. 5A to 5D are scanning electron microscope images of coated patches from different coating solutions of low viscosity (i.e. <<1 Pa S) as applied to the projections of FIG. 4A, whilst FIGS. 5E and 5F show low viscosity coating of projections similar those of FIG. 4B having a length of 60 μm. The coating solution contained methylcellulose (viscosity enhancer), Quil-A (surfactant) and OVA protein (active agent), and had a viscosity in the region of 55 mPa·S. Specific coating solutions are set out below. The patches were dipped into the solution for 10 seconds and dried in air for 1 hour. The morphology of the coated patches was then observed by SEM.

A representative result is shown in FIGS. 5A and 5B. From these figures, it can be seen that a very thick coating (>10 μm) has been formed on the base of the patch. The other four patches were dipped into the same solution in the same way, but repeating 3 cycles of the dipping process. The result is shown in FIGS. 5C and 5D. It can be seen that the coating thickness on the base of the patch greatly increases after 3 dipping cycles, with almost half of the projections being covered by the coating on the base. These data show that dipping the patches into a solution of low viscosity leads to a thick vaccine coating on the base of the patches, which is not optimal, because the base of the patches can not be inserted in the skin and, correspondingly, the coating on the base can not be delivered to the skin.

Thus, these results demonstrate that dip coating does not work properly for these patches if the viscosity of coating solution is very low (<<10 Pa.S). More specifically, very thin or no coating has been obtained on projection tips, whilst the majority of coating appears on the base of patches. The reason is that when the densely packed projection patches contact coating solution, the solution will form a drop and cover all projections due to the huge capillary action. This results in a long drying process, during which the coating solution slowly drips onto the base of the patches. Coating on the projections is therefore thin (<0.2 μm) whilst multiple-dipping cycles lead to a very thick coating on the base of patches, but not on the projections as required.

For the purpose of these examples, the coating solution is composed of, for FIGS. 5A and 5B, Methylcellulose (MC) 10 mg/ml, Quil-A 2 mg/ml, OVA 5 mg/ml; for FIGS. 5C and 5D, MC 30 mg/ml, Quil-A 2 mg/ml, OVA 5 mg/ml; and for FIGS. 5E and 5F, Carboxylmethylcellulose (CMC: Mw of 90 kDa, 250 kDa, 700 kDa) 1% or MC (35-55 m Pa.S) 1-2%, poloxamer 188 0.5%-2%, DNA 1-2 mg/ml. The patches were coated by dipping projections into coating solution and then withdrawn to dry in air, for FIGS. 5A, 5C, 5E or vacuum, for FIGS. 5B, 5D, 5F.

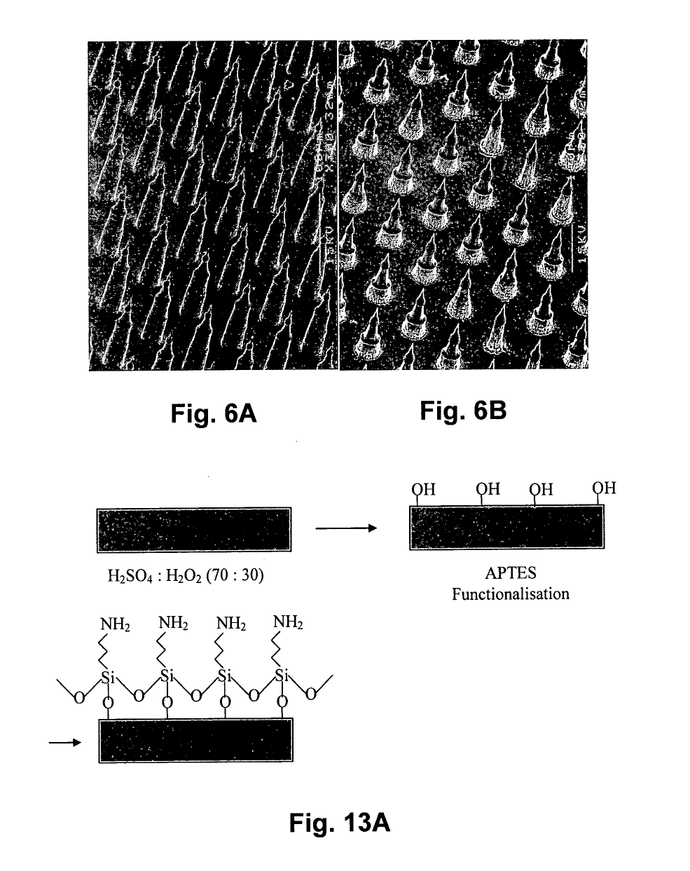

FIGS. 6A and 6B are SEM image of coating on projections from a coating solution (coating solution: 6.5 ml H₂O, 0.7 g Pectin, 13.8 g Sucrose, 5 mg Quil-A, 2.5 mg/ml Rhodamine-Dextran). These images show the coating has been achieved on the tips only without contaminating the patch base and the shaft of projections. During the drying process of coating solution on the tips, the coating solution tends to form a ball to reduce its surface energy. This is influenced by the curvature of the tips, and it will be appreciated selection of an appropriate curvature can assist in controlling this effect. In some examples, the effect may be desirable to ensure a large volume of payload on the projection tips, whereas in other situation, its reduction may be desirable to avoid using excess material when coating the tips. The thickest coating is up to 1.8 μm in one dipping cycle.

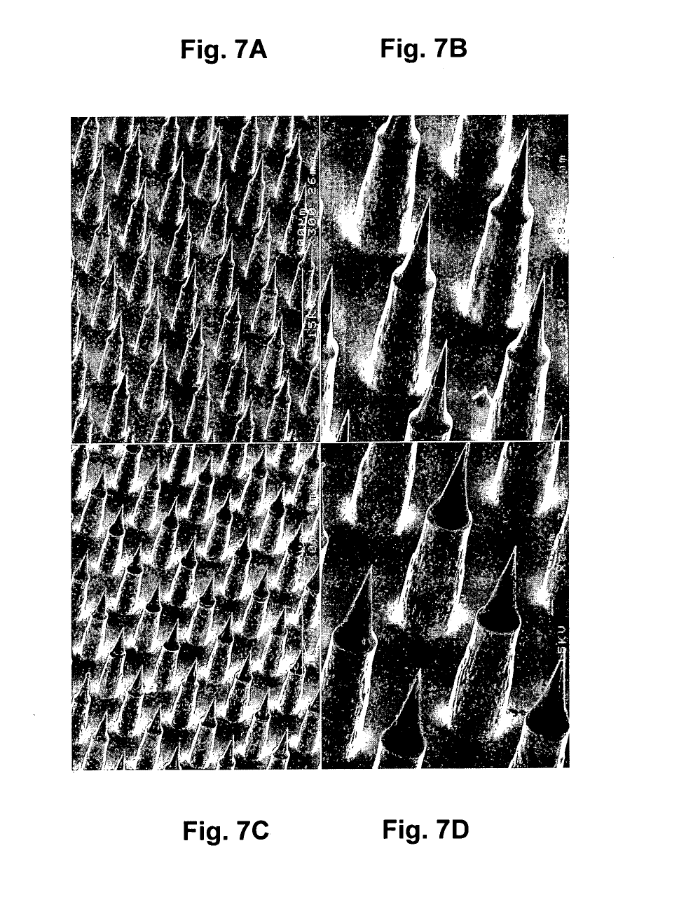

FIGS. 7A to 7D are SEM images of coating on projections from a coating gel (coating solution: 6.5 ml H₂O, 0.7 g Pectin, 13.8 g Sucrose, 5 mg Quil-A, 10 mg/ml DNA, allowed 2 days set at 4° C., viscosity: ˜4.5 Pa.S, when measured at a temperature of 23° C. and a shear strain rate of 1 sec⁻¹. The coating area provides a dark signal while the uncoated area shows much brighter signal. Accordingly, FIGS. 7A and 7B show that the top 20 μm of the projections have been coated with DNA, whilst FIGS. 7C and 7D show the whole upper conical part of the projections (top 40 μm) coated with DNA. The coating thickness is 1.2±0.1 μm (n=30 projections on 3 patches). From these images, it can be seen that coating can be controllably applied on the top part of the projections (i.e. both in terms of coating thickness and coverage length), with the coating length being controlled by a depth of immersion (or dipping depth) of the projections in the coating gel.

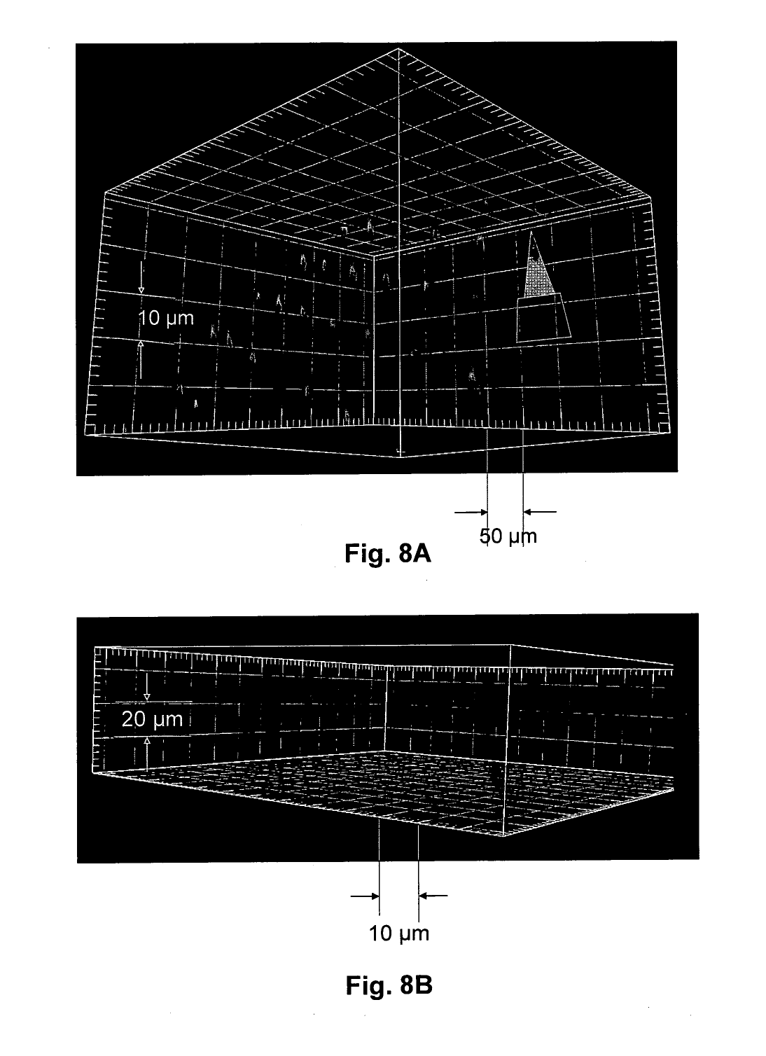

With controllable coating of projections established, the next example was used to demonstrate that biologically active (or relevant) material was uniformly coated on the projections, i.e., not only other excipients, such as, viscosity enhancers, surfactant, appeared on projections after coating. FIG. 8A shows the fluorescence microscopy images of projections after being dip coated by rhodamine-dextran. The fluorescent dye coating can be clearly observed. As shown, the fluorescent dye has been controllably coated onto the top 20 μm area only of the projections, with no coating on the base of the patch. This highlights that the biologically active (or relevant) material was uniformly coated on the projection tips, and that the coating was not limited to other coating solution excipients, such as, viscosity enhancers, surfactants, or the like.

FIG. 8B is a confocal microscopy image of fluorescent dye released from coated projections. This image shows that the coated Rhodamine-Dextran can be delivered up to 30 μm under the ear skin of a C57BL/6 mouse, thereby demonstrating the effectiveness of the patch at delivering material to a subject. This depth of penetration coincides with targeting cells within both the skin epidermis and dermis, of the mouse ear—and is consistent with original targeting embodiments described in copending U.S. patent application Ser. No. 11/496053.

The above described examples have focused on the projections of FIG. 4A. Accordingly, further examples were performed using the projections of FIG. 4B.

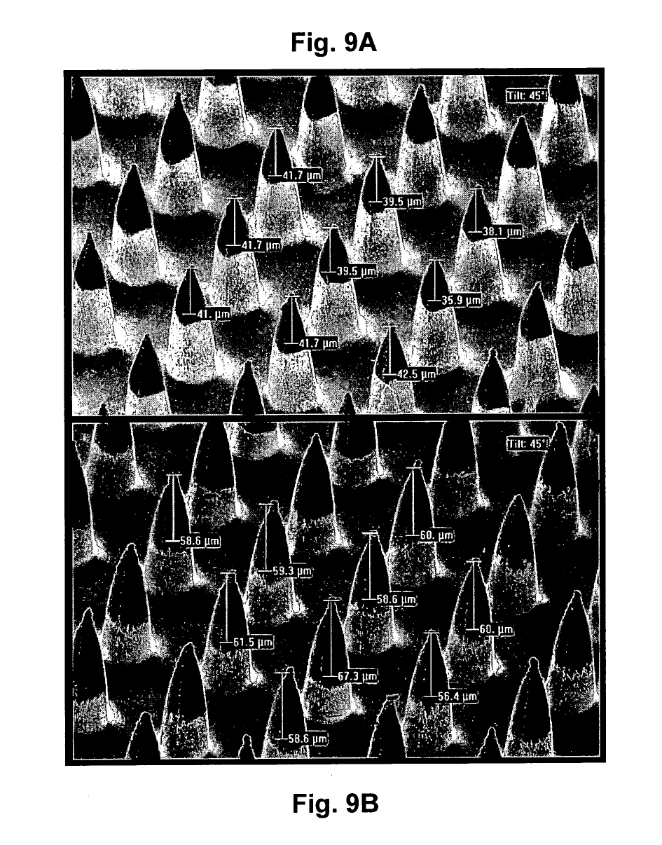

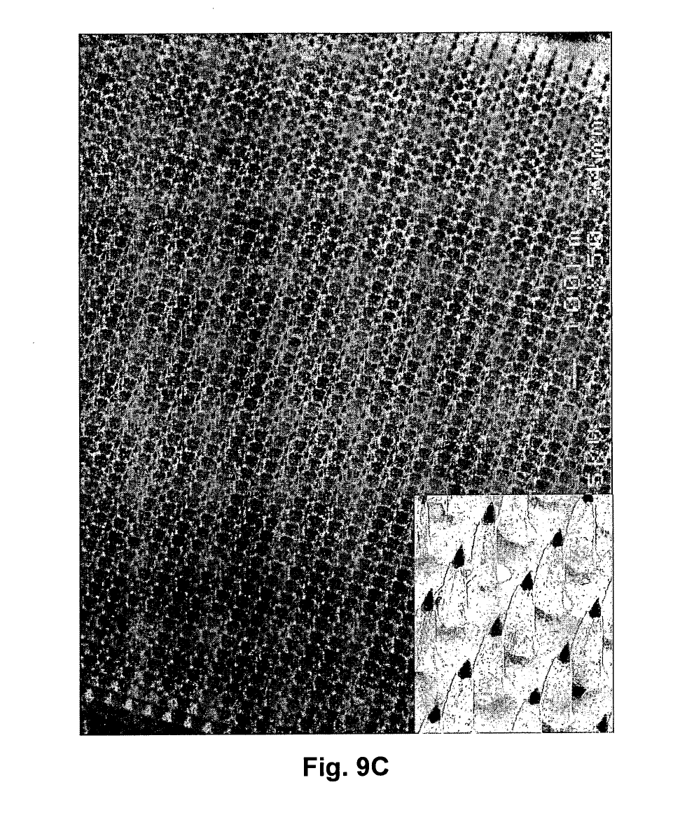

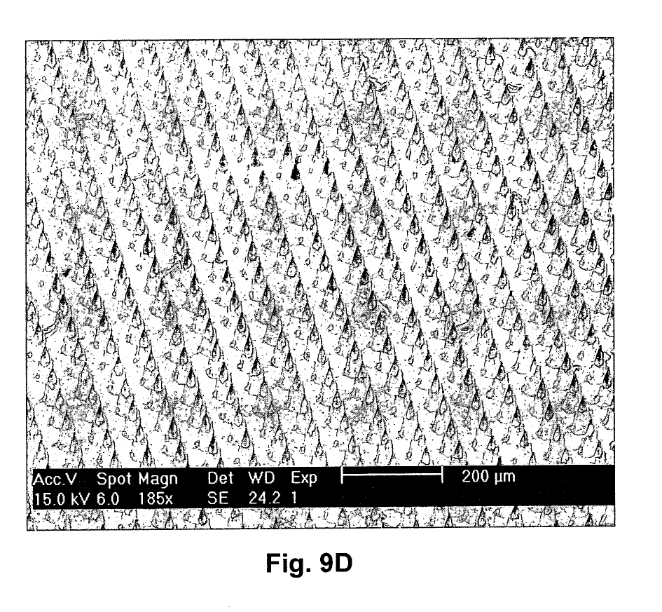

FIG. 9A shows an image of coated projections, with only the top 40 μm coated with OVA protein. FIG. 9B is an image of coated projections, with the top 60 μm coated with OVA protein. The coating lengths of projections in both images have been shown to demonstrate the consistency of coating on each projection. In FIG. 9A, the top 40.2±2.0 μm (average±standard deviation) has been coated. In FIG. 9B, the top 60.0±2.9 pm (average ±standard deviation) is coated by OVA protein. This technique can achieve uniform coating on the tips of thousands of projections in a large area.

By coating only the tips of the projections, this ensures that coating material is only provided on the portions of the projections that penetrate within the skin to thereby deliver active material to a desired region in skin. By contrast the remaining portion of the projections, and the base of the patch remain uncoated. This maximises the proportion of active material effectively delivered to the subject, reducing wastage of coating solution and hence reducing the dose of any active material required to obtain a response from the subject. This reduces the cost of coating the patches as well increasing the number of subjects that can be treated with a given amount of material, which is important in mass vaccination situations, or when supplies of active material are otherwise restricted.

Further examples are shown in FIGS. 9C and 9D in which coating is applied uniformly to thousands of protections, thereby demonstrating that the technique works evenly across the entire patch and is not a localised effect.

In the above examples of FIGS. 6 to 9, the patches have been coated in a highly viscous solution, typically having pectin as a viscosity enhancer. An examples of the use of sodium alginate as part of a coating solution will now be described. In this example, the coating solution was composed of 20 mg/ml of sodium alginate, 600 mg/ml of sucrose, and 10 mg/ml of OVA protein, having a viscosity of 7.0 Pa.S, with the resulting coating being shown in FIG. 10. In this example, the patch surface does not have a thin layer of gold coating, so the contrast between OVA protein coating and the silicon surface of the projections is not high. Despite the lower imaging contrast, it is still clear that the distal half of the projections have been uniformly coated.

It will be appreciated that this highlights that the immersion techniques described above allow coatings to be reliably provided on the tips of projections, without relying on material surface chemistry of the projections to control how well the coating approach works. This allows coatings to be reliably applied to projections having different surfaces such as gold or silicon.

To perform a further quantitative study of the bulk released payload within skin following patch application, a Coomassie Blue R 250 dye was used. In this example, the coating solution contained 20 mg/ml of sodium alginate, 600 mg/ml of sucrose and 10 mg/ml of Coomassie Blue, and had a resulting viscosity of 7.0 Pa.S, when measured at a temperature of 23° C. and a shear strain rate of 1 sec⁻¹. On six patches in total, the measured coating quantities on each patch totalled 172.0 ng ±21.0 ng, thereby highlighting adequate coating of the projections.

The skin was then examined to determine where the payload was delivered within the skin, relative to key skin strata. To do this, rhodamine-dextran coated patches were applied to the mouse ears and examined with imaging methods to assess the success of the payload delivery. For this particular study, the conditions were: the coating solution was composed of 20 mg/ml of sodium alginate, 600 mg/ml of sucrose, and 5 mg/ml of rhodamine-dextran. C57BL/6 mice were anesthetized and their ears were placed on a 3 mm thick vinyl pad to serve as a cushion. Each patch was applied to the inner earlobe with a velocity of 2 m/s and a force of 0.6 N by an applicator device. Each patch was delivered to the inner earlobe. The patch was then held in place for 5 seconds to ensure a complete release of coating in the skin. The procedures described herein were approved by the University of Queensland Animal Ethics Committee.

Mice were subsequently sacrificed and ears excised and fixed within 30 minutes of patch application. The ears were fixed in 2% paraformaldehyde in 0.1M phosphate buffer overnight at 4° C. The ears were rinsed for 3×10 minutes in 0.1M phosphate buffer. The ears were then separated, isolating the patched side consisting of the dermis and epidermis. The tissue was then permeabilised using a stock solution of 0.25% Triton-X in TBS for 30 minutes. The ears were rinsed for 3×10 minutes in TBS buffer. A solution of 5% sterile filtered BSA in TBS was used to block the tissue for 1.5 hours. A working solution of MHC-II conjugated FITC was made by a 1:50 dilution of 0.5 mg/ml MHC-II FITC in 1% BSA in TBS. The tissue was stained for 2 hours at room temperature with the working solution of MHC-II FITC. The tissue was rinsed for 3×10 minutes in TBS. A stock solution of 10 mg/ml Hoechst 33342 in DMSO was prepared. A working solution was made by a 1:10,000 dilution in 1% BSA in TBS for nuclei staining. The tissue was treated with the working solution of Hoechst 33342 solution and incubated for 30 minutes at room temperature. Finally, the mouse ear sections were rinsed for 3×10 minutes in TBS.

A series of images at successive skin depths were taken under confocal microscope (Zeiss 510 Meta, Germany) on the fixed 4 ears to observe the coating delivery in the skin. In order to know the colocalization of delivered material and MHC-II cells, the coating delivery was analysed in 5 positions (the centre and four corners of the patched area) on each mouse ear (4 ears in total). In each position, 144 delivery sites were investigated.

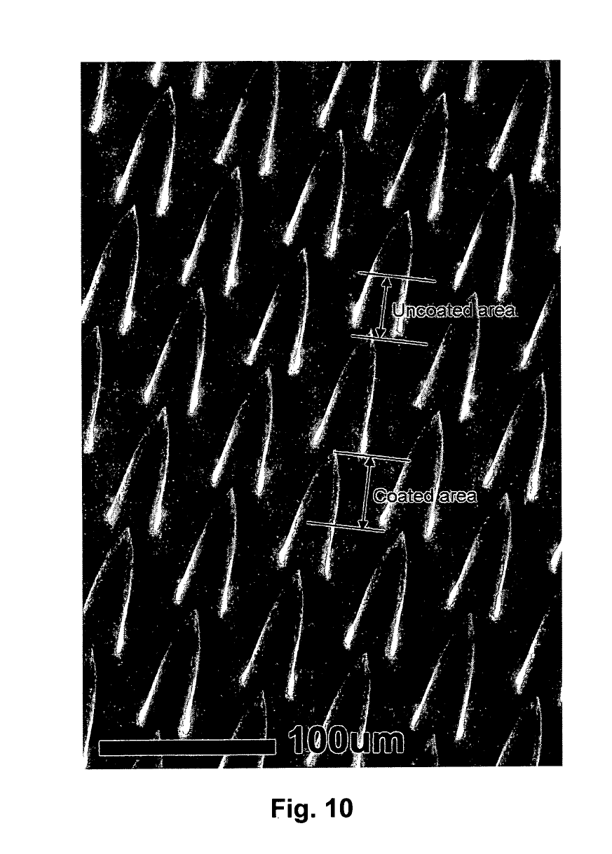

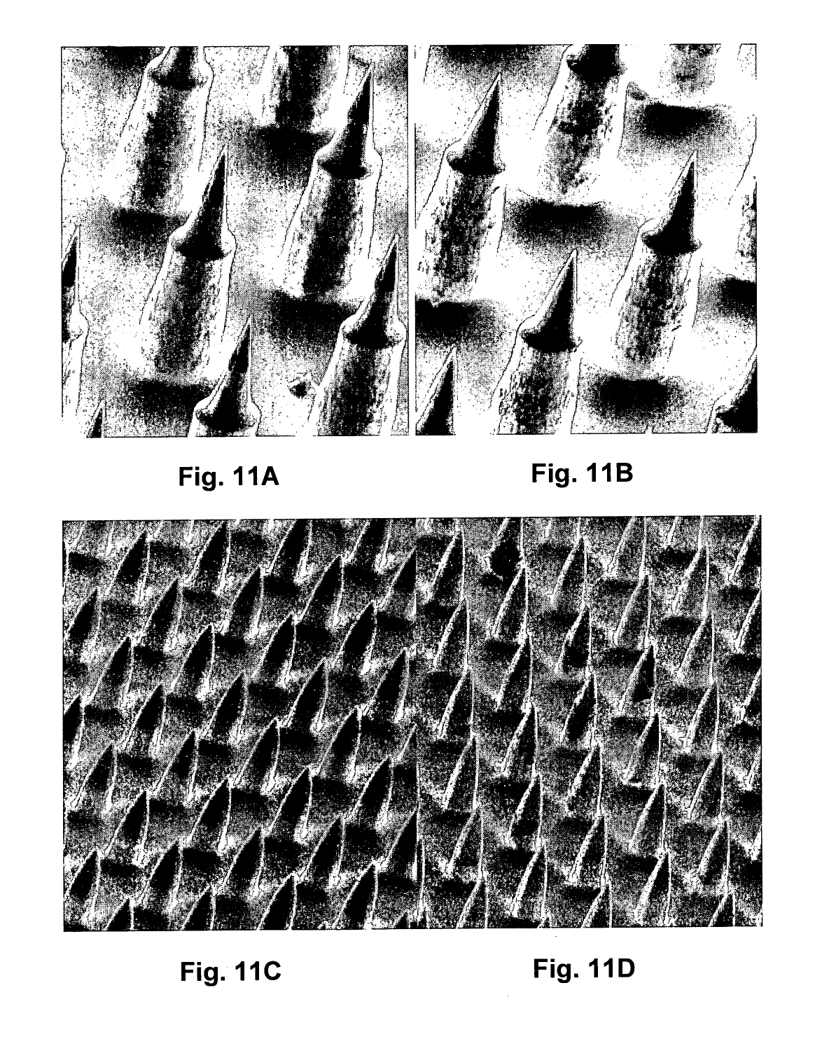

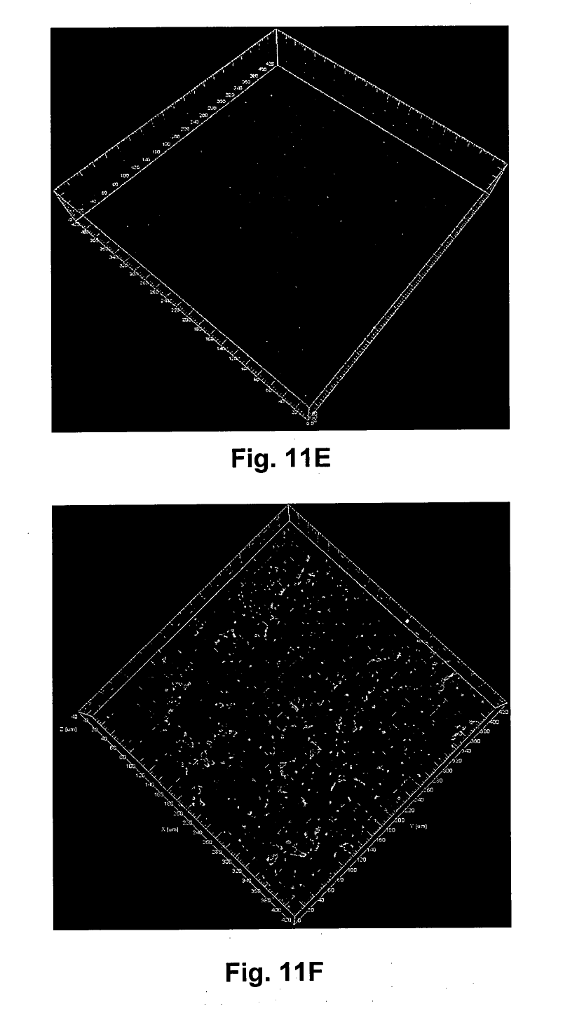

FIGS. 11A and 11C show the coated projections before being applied to mouse ears, with FIGS. 11B and 11D showing the projections after application. It can be seen from FIGS. 11A and 11C that the projections have uniform coating before being applied to the mouse ears. Then, as desired, after those projections were applied to the mouse ears, the uniform coating was rapidly removed, confirmed by the SEM images shown in FIGS. 11B and 11D. Some areas showing dark signal on the projections in FIGS. 11B and 11D may be remaining coating or contamination from the application of the patches to the mouse ears. To rule out the possibility of incomplete release, the patches were observed under fluorescence microscope. Before patch application, the coated projections show fluorescence, indicating that the coating of rhodamine-dextran was achieved, as shown in FIG. 11E. However, by contrast, after application, no fluorescence was observed, indicating that rhodamine-dextran was no longer present on the patch, and hence had been delivered to the subject.

Through the above experiments, it is demonstrated that the coating has been removed during patch application. However, it is not clear that whether the coating has been delivered in the skin or it has been wiped off during the insertion of the coated projections into the skin. To clarify this, six rhodamine-dextran coated patches were applied to the skin and then the mouse ears were stained for observation under fluorescence microscope. A series of images at successive skin depths of the mouse ear were taken to show the delivery of the coating in the skin.

FIG. 11F shows a snapshot of a 0.176 mm² region of the patched area with a total of 36 projections sites. A series of combined z-stacks were obtained to form a 3-dimensional image. MHC-II positive cells were stained using a MHC-II conjugate-FITC stain. The second harmonic generation of collagen in the upper dermis is also shown. From FIG. 11F, it can be seen that the rhodamine dextran coating is released in the skin within 5 seconds. According to analysis, 61.1±12.2% of the coating delivery sites colocalised with MHC-II positive cells within the viable epidermis (indicated by the arrow heads and the inset).

The above results (FIG. 11) therefore show that the coated rhodamine-dextran, a surrogate of vaccines (or other drug/immunotherapeutic), is effectively and rapidly delivered to the skin, and in particular to the skin strata and cell types as outlined in copending U.S. patent application Ser. No. 11/496053. In order to determine the coating delivery efficiency (mass delivered to the skin vs mass coated on the skin), 4 Coomassie Blue coated patches were applied on 4 mouse ears by custom based applicator device. To achieve this C57BL/6 mice were anesthetized and their ears were placed on a 3 mm thick vinyl pad to serve as a cushion, with each patch being applied to the inner earlobe with a velocity of 2 m/s and a force of 0.6 N. The patch was then held in place for 5 seconds to ensure a complete release of coating in the skin. To determine the amount of Coomassie Blue transferred to the skin surface, skin sites were cleaned with a Scotch tape. Used tape was soaked overnight in 150 μl of 70% ethanol to elute the Coomassie Blue. Used patches were also soaked in 150 μl of 70% ethanol to determine how much coating remained on the patches after application. The solutions were centrifuged at 10,000 g for 5 minutes to remove the debris from mouse ear skin. Then the Vis absorption spectra of all the solution samples were scanned. The absorbance (@592 nm) of all samples was recorded and compared with the absorbance of standard samples, so the amount of coated vaccine on each patch can be calculated. Using a mass balance, the amount of Coomassie Blue delivered into the skin was determined by subtracting the amount remaining on the projections and on the skin surface after insertion from the amount originally on original coated projections.

The measured amounts of Coomassie Blue coated on the patches, left on the mouse ears, and remained on the patches after application were 172.0±21.0, 13.3±2.4, 9.2±1.6 ng, respectively. Using mass balance, the calculated coating delivery efficiency is about 86.9% in mass. In other words, 86.9% of the coating on the projections was delivered to the skin—a highly desirable outcome, as it minimizes wastage of the coated active ingredients. This is a major advance over existing methods.

Overall, these results show that the coated projections robustly pierce the skin, with a subsequent, rapid release of coating delivered within the target skin layers. In summary, the coating is successfully delivered into the skin by coated projection patches, using the above described coating approach.

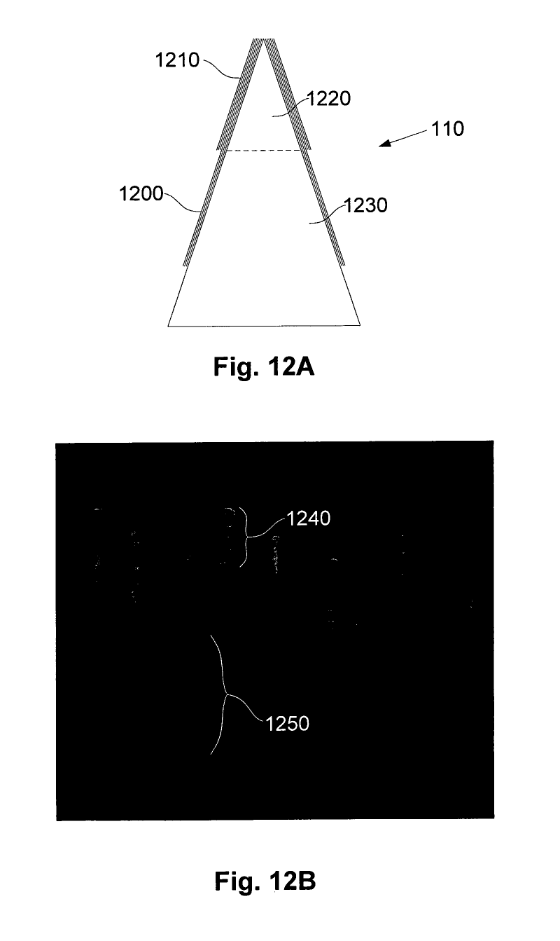

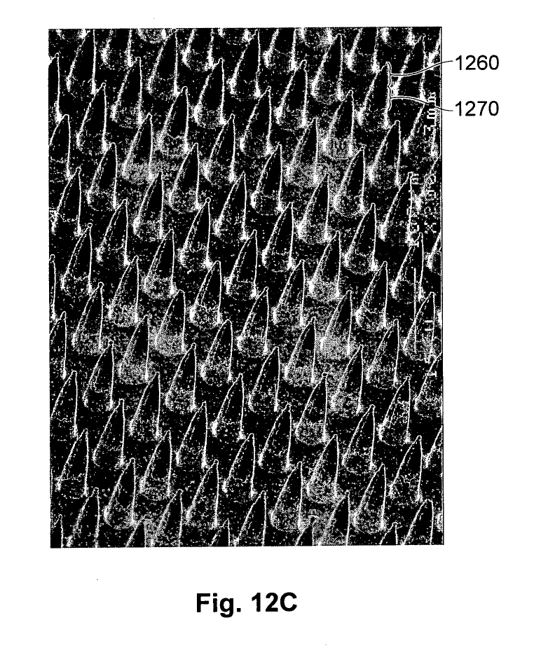

An experiment involving the provision of multiple coating layers will now be described with reference to FIG. 12A, which shows an example of a single projection coated with multiple layers.

In one example, a patch having a plurality of projections 110 is coated with one layer 1200 of FITC-Dextran. This was achieved by performing the above described coating process, allowing the coating to dry, and then repeating this until three layers were established. Following this, second layer 1210 of Rhodamine-Dextran was coated on the top of FITC-Dextran. In this example, the second layer 1210 has a shorter length than the first layer 1200 so that a tip part 1220 of the projection 110 has both FITC-Dextran and Rhodamine Dextran coating, whilst a bottom part 1230 had only FITC-Dextran. However, other coating protocols can be used, such as using a shorter dipping depth, to form a first layer of coating, which was then dried by a nitrogen jet for 1 minute, with a second dipping process at a much longer dipping depth being used to thereby form a second layer of coating. FIG. 12B is an example of a Multi-Photon Microscope (MPM) image showing the fluorescence from FITC-Dextran and Rhodamine-Dextran coated on projections coated as described above with respect to FIG. 12A. The fluorescence of FITC-Dextran is indicated at 1240, whilst the fluorescence of both FITC-Dextran and Rhodamine-Dextran on the tip part 1220 of is shown at 1250.

An example SEM image is shown in FIG. 12C. In this example, the top part of projections provide dark signal because of the thick coating while the middle part has brighter signal due to the thin coating. Moreover, the bottom area and the base of patches have the brightest signal since there is no coating at all. This highlights that the tip part has thick coating 1260 and the bottom coating is thinner 1270. Thus, coating the projections in this manner leads to a coating that is thick on the tip of the projections as this part of the projections is dipped into the highly viscous a greater number of times that the middle part of the projections, where the coating is therefore thinner. This confirms the controllability (i.e. both the coating length on the projections and its thickness) of the coating process.

A patch coated in this manner was then used to deliver material to a mouse ear, using the techniques outlined previously. MPM images showing the delivery of material to the mouse ear are shown in FIGS. 12D and 12E. This indicates not only that FITC-Dextran (green) and Rhodamine-Dextran (red) have been delivered to mouse ear skin, as shown in some instances at 1280, 1290, but also that some delivery sites have mixed FITC-Dextran and Rhodamine-Dextran (indicated by white arrows 1295).

Accordingly, this experiment demonstrates not only that the coating thickness and length can be controlled using the above described coating techniques, without using a physical plate with dipping holes which requires time-consuming alignment, but also that the resulting coating can be successfully delivered to a subject upon suitable application of the patch.

A further experiment was performed to determine the impact of surface modification of the projection patch on the coating methodology, and in particular, the surface modification through the use of a hydrophilic material. The surface modification process makes the projection surface more hydrophilic and smoother compared with the projections without being surface modified. Therefore, the coating thickness and payload are increased relative to if the projection surfaces were unmodified.

In a first example, the patch surface was modified by pre-coating the patch with 1% methylcellulose on the patch using the gas jet-drying coating technique described for example in copending patent application number WO2009/079712. Following this the patches were dipped into a coating solution, containing sodium alginate and Coomassie Blue, to cover the tips of the projections only. Quantitative results of the coating process are outlined in Table 1. The results show the payload of coating obtained from the dip coating process (not from the pre-coating, because pre-coating solution did not contain Coomassie Blue).

TABLE 1

| | |
|---|---|
| Coating Solution | Results |

| MC (%) | Na Alginate (%) | Mean (μg) | SD |
|---|---|---|---|
| 1 | 1.5 | 0.613 | 0.148 |
| 1 | 2.5 | 0.495 | 0.138 |
| 0 | 2.5 | 0.123 | 0.080 |

Table 1 highlights that the coating payload can significantly increase if the patch has been pre-coated with a layer of MC, and in particular, that the payload can be as high as 4 times that if the patch has been simply coated with one layer of MC.

In a second example, Thiol modification of a patch is achieved using an APTES (3-aminopropyl triethoxysilane) solution that is commonly used to react with glasses and siliceous surface, such as a silicon wafer based patch, to thereby cause the formation of a aminopropyl substituent on the surface of the patch, which in turn results in a hydrophilic surface.

In this example, the protocol for producing the patches is as follows:
1). Wash patches with piranha (3 portions of H₂SO₄ and 1 portion of H₂O₂ in volume) solution;
2). Rinse (water, methanol, 1:1 methanol/toluene);
3). Soaked in 10% APTES solution in toluene overnight under N₂;
4). Rinse with ethanol, 2-propanol and water;
5). Dip the surface modified patches into a coating solution, containing sodium alginate, sucrose and Coomassie Blue, for site-selective coating.

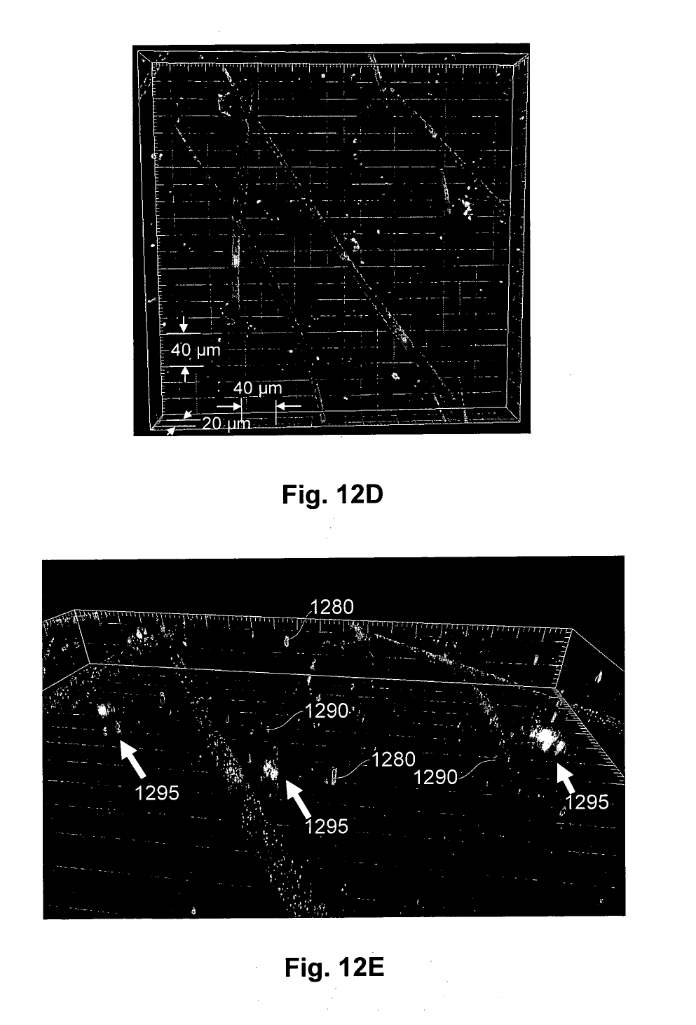

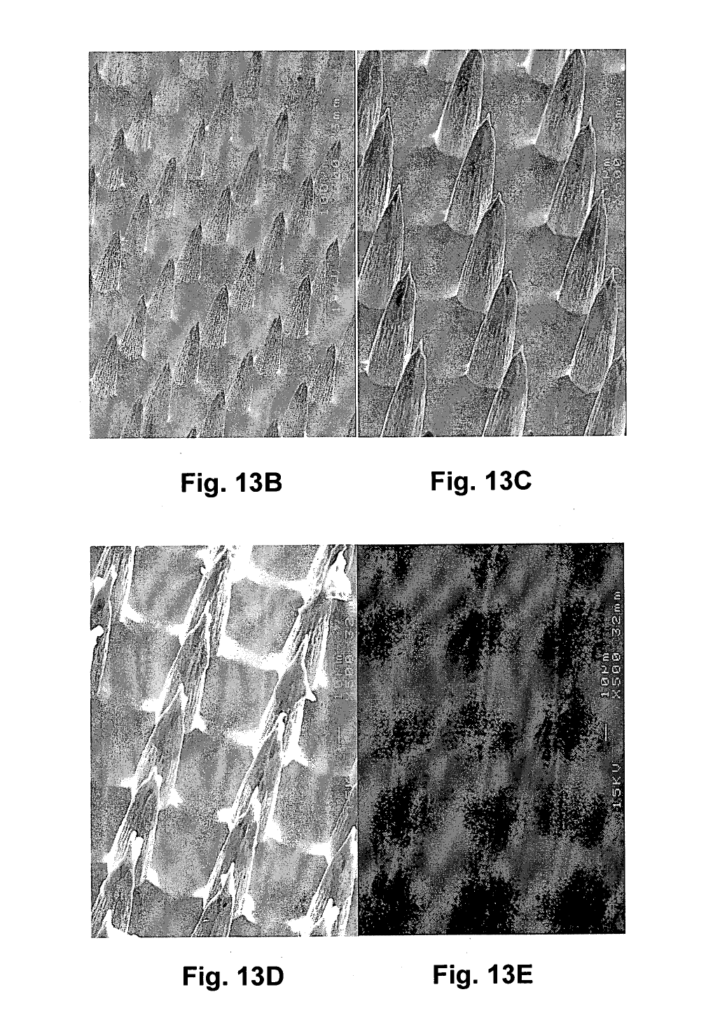

FIG. 13A illustrates schematically the patch surface modification scheme by APTES.

FIG. 13B shows the coating achieved for a patch that has not been treated with APTES and which is coated using sodium alginate, Coomassie Blue and sucrose. To illustrate the benefits of surface modification, patches were chosen with a very rough initial surface on the projections. The coating applied is a think coating and the microstructures on the original projections can still be clearly observed after the thin layer coating has been applied.

FIG. 13C shows an APTES treated patch that has been coated once in a coating solution, containing sodium alginate, Coomassie Blue and sucrose. Compared with FIG. 13B, the coating thickness has significantly increased to around 2 μm. The rough surface structure of the projections has been fully covered by the coating.

FIG. 13D shows an APTES treated patch that has been coated twice in a coating solution, containing sodium alginate, Coomassie Blue, and sucrose. Compared with FIG. 13C, the coating thickness has further dramatically increased to around 5 μm.

FIG. 13E shows a backscattered electron image of the coated patch of FIG. 13D. The thickly coated part shows dark signal, which qualitatively confirms the thick coating on the projections. This demonstrates that surface modification increases the coating thickness significantly, from ˜100 nm to over 2 μm thickness, which allows the coating payload to be greatly increased by modifying the patch surface with hydrophilic compounds.

In a further example, vaccine was delivered to demonstrate the ability to induce a systemic immunological response. In this example, the vaccine used was a seasonal human anti influenza vaccine, Fluvax2008®, manufactured by CSL Ltd, Parkville Australia which contained 15 ug haemagglutinin each of the following strains of influenza per 0.5 ml: A/Brisbane/10/2007 (H₃N₂), A/Solomon Islands/3/2006 (H₁N₁) and B/Florida/4/2006.

Each patch was pre-coated with MC followed by dip-coated with commercially-available trivalent influenza vaccine (Fluvax2008® CSL Ltd, Parkville Australia) using the method described above. The coating solution contained 20 mg/ml of sodium alginate, 600 mg/ml of sucrose, 360 μg/ml of influenza vaccine, and 10 mg/ml of Quil-A.

Using an applicator device, each patch was delivered to the inner earlobe of anaesthetised female 6-8 week old C57BL/6 mice (housed in a specific pathogen free environment) with a velocity of 1.9 m/s and a force of 0.6 N. After application, each patch was retained in place on the skin for 2 minutes, to give adequate time for the vaccine to dissolve and diffuse into the skin epidermis/dermis. A range of doses were tested across different experimental groups.

Additional groups of mice were vaccinated by needle and syringe in the caudal thigh muscle. The doses shown are the total HA amounts delivered under the skin of the three different strains present in Fluvax®. Following the single vaccination, all mice were bled at 3 weeks. The sera separated and stored frozen at −20° C. till assays were performed.

ELISA was performed, with the ELISA plates (Nunc Maxisorp) being coated with commercial trivalent split virion Fluvax2008® at a concentration of 3 μg/ml total haemagglutinin in 0.1M sodium bicarbonate buffer overnight at 4° C. and were used to determine the titres of specific IgG elicited. The colour development was performed using ABTS (2,29-azino-bis[3-ethylbenzthiazoline-6-sulfonic acid]) (Sigma cat. no. A-1888) as the substrate. The absorbance readings at 405 nm were measured against control wells containing no antiserum in the reaction. Each sample was individually analysed.

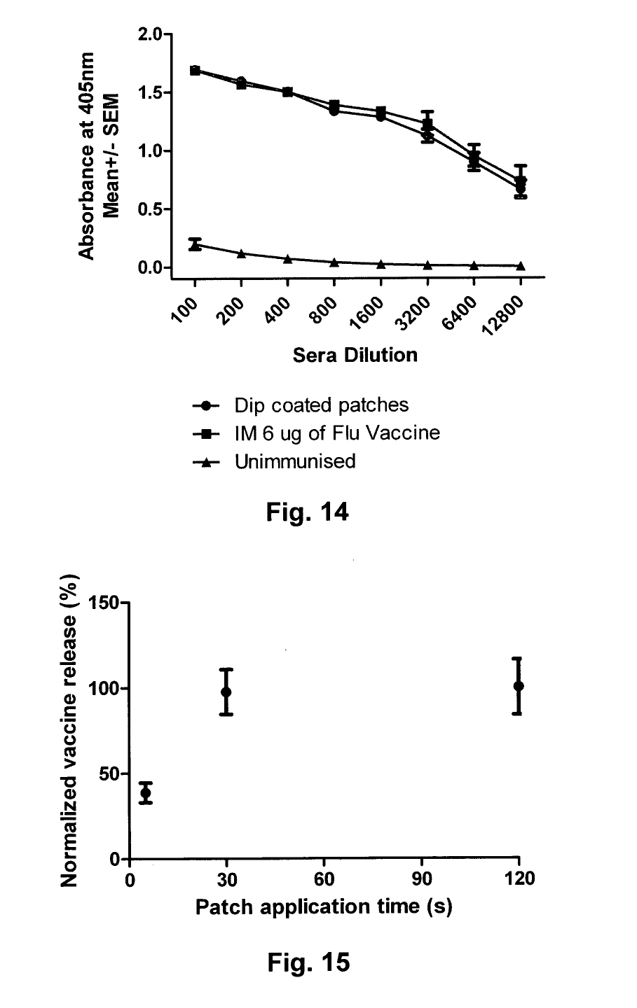

FIG. 14 is a graph of an example of the systemic immune responses induced by influenza vaccine delivered by patches and intramuscular injection. The dip coated patches containing flu vaccine can induce comparable antibody IgG titer with the mice immunised by intramuscular injection of 6 μg of flu vaccine.

A further example relating to the release kinetics of ¹⁴C OVA immersion-coated patches will now be described. In this example, each patch was dip-coated with ¹⁴C OVA for only one dipping cycle. The coating solution contained: 20 mg/ml of sodium alginate, 600 mg/ml of sucrose, 165 μg/ml of ¹⁴C OVA, and 10 mg/ml of Quil-A.

The patches were applied to the inner earlobe of anaesthetised female 6-8 week old C57BL/6 mice (housed in a specific pathogen free environment) using an applicator device, so that each patch was delivered with a velocity of 1.9 m/s and a force of 0.6 N. After application, each patch was kept in place on the skin for 5 s, 30 s or 2 mins, to explore the release kinetics of coated ¹⁴C OVA. For each application time (5 s, 30 s and 2 minutes), a group of 5 coated patches were applied on 5 individual ears for measurement of the released amount of coating in the skin.

Following application, the skin sites were gently and thoroughly cleaned with a cotton swab imbibed with physiological saline immediately following patch removal. Subsequently, the skin was cut and then collected in the scintillation vials containing 1 ml of PBS and mixed. Each ear was placed in an 1.5 ml Eppendorf tube containing 750 ul of tissue solubiliser solution (Soluene-350 Perkin Elmer). Then the tubes were sealed and heated in a heating block at 60° C. for 2-4 hours to solubilise the tissue. The samples were allowed to return to room temperature and the contents were transferred to a liquid scintillation. 10 ml of Scintillation liquid (Hionic-Fluor Perkin Elmer) were added to each vial. All vials were counted in a liquid scintillation counter for 2 min each sample to determine the amount of ¹⁴C OVA delivered to the skin.

The results of the release analysis are shown in FIG. 15. These data show that the released amount of ¹⁴C OVA in the skin does not show statistical difference for patch application times of 0.5 and 2 minutes.

Accordingly, the above described experiments highlight how coating the projection patches with a low viscosity solution, such as a methylcellulose solution having a viscosity of 55 mPa·S, results in significant coating obtained of the base of the patches, with reduced coating of the projections. This is caused by the coating solution gathering at the base of projections due to capillary action. Therefore, the base of the patches was always contaminated and it is very difficult to control of the coating length on the projections without using a physical mask with “dip-holes” to eliminate the effect of capillary action.

The high volume of coating solution on the patch base has a number of drawbacks. For example, the patch base does not penetrate the subject, and hence any material contained in coating solution on the patch base will not be delivered to the subject, and as a result is wasted. Additionally, the coating solution forms a layer on the patch acting as a physical barrier to penetration of the projections into the subject, thereby further reducing material delivery. It is therefore desirable to provide coating processes that eliminate the coating on the base of the patches and have better control of dip/coating length on each projection.

The experiments highlight that dip-coating in a solution of high viscosity can selectively coat materials onto the projections. In particular, selecting a highly viscous coating solution, containing a suitable viscosity enhancing agent such as methylcellulose, sodium alginate, or the like, this has higher resistance to shear or displacement stress, and hence reduces the rate of capillary induced movement of the coating solution, thereby allowing the length of projection that is coated to be easily controlled. As a result, predominately only the part of the projections dipped in the coating solution will be coated and thus the coating length can be controlled to a greater degree.

The required coating length can be determined by how much penetration depth the projections are required to make in skin, with only the coating on the area which can be inserted in skin releasing vaccine in the skin for inducing immune responses. Therefore, coating material only on parts of the projections that will penetrate the subject, with no coating on the remainder of the projections and patch substrate, this can substantially minimize wastage of expensive vaccines, drugs, or other materials.

Additionally, selection of suitable viscosity enhancing agents, such as pectin and sodium alginate, are all approved by the Food and Drug Administration (FDA) in USA for human use. Therefore, our coating technology potentially satisfies human use regulatory requirements.

The data also show the method uniquely achieves uniform coating of a wide variety of materials to specified sections of very small and densely packed projections. These materials can include, but are not limited to classes of conventional vaccines (e.g. OVA protein; MW: 44287 Da), DNA, and rhodamine-dextran (2 MDa).

The coated projections are also robust enough to pierce the skin and the coating shows very fast release (within 5 seconds) into the skin. The coated rhodamine-dextran (a surrogate of vaccine) can be directly delivered to antigen-presenting cells or to the area around these cells, with a coating delivery efficiency as high as 86.9%. Collectively, this demonstrates the desired delivery characteristics in skin are successfully achieved.

Conceptually, the payload coated onto each patch is a function of several parameters, including the:
coating thickness on each projection;
size of projections and patches;
number of projections and patches;

Furthermore, the coating can be optimised by selection of a viscosity to reduce capillary action, which in turn can depend on the contact angle or hydrophilicity of the patch. The contact angle will typically depend on patch properties, such as the projection geometry and/or spacing, as well as any other projection properties, such as surface modifications, including the presence of any coatings or the like.

In one example, successful delivery of material is demonstrated using patches having 3364 projections, spaced and distributed on an applied surface of 16 mm², coated with 173 ng Coomassie Blue with a thickness of around 1 μm. Typical surface areas of standard patch devices already used in humans (by others) are much larger, extending into cm² range and the coating thickness is up to 10 μm. With these parameters, the total coating amount with projections can extend into sub-milligram range, whilst still being capable of producing an immunological response when delivered to immune cell populations.

Accordingly, this demonstrates that a simple dip coating technique in solution of high viscosity allows active materials to be coated on projections at a controllable length on selective site, with no coating on the base. By doing this, the coating delivery efficiency reaches up to 86.9%. This technique can coat a wide variety of molecules, including OVA protein vaccine, DNA, and fluorescent dyes, on projection patches. Following application, the projection patches are able to quickly deliver coating into the skin (within only 5 seconds). This technique has the potential for scaling up to coat a massive number of patches for vaccine delivery to human in the future.

The above described techniques can therefore achieve uniform and controllable coating onto the tip of small and densely packed projections. The coating can avoid contamination of the base of patches and the bottom part of projections that can not be inserted into skin for coating delivery. The materials coated on the projection tips can achieve fast delivery, within seconds, the skin, or targets therein such as highly immunologically sensitive cells within the epidermis.

This technique can be used for, but is not restricted to use with vaccines. For example, drugs can be coated onto projection tips for drug delivery. Alternatively immunological adjuvants, virus like particles, or the like, can be coated onto projection tips for delivery.

In one example, the projections are dipped into a coating gel and then withdrawn to form a dry coating in air or under a gas jet for fast drying. The coating gel has a viscosity of 4.5 Pa.S, so capillary action will not occur during dipping process. In another example, projections are dipped into a very viscous coating solution and then withdrawn to form a dry to coating in air or under a gas jet for fast drying. The coating solution has a viscosity higher than 1 Pa.S, so capillary action will not happen if the dipping process is in a short time. The coating length can be controlled by the dipping depth in the coating solution.

The coating length can be controlled by using a positioning system, or preparing the coating solution or gel as a film, no thicker than the length of the projections, provided on a superhydrophilic surface (in which the contact angle of coating solution on the surface is less than 5°), allowing the coating length to be controlled by the thickness of the film.

The coating length can be controlled electronically for mass production by utilizing the electrical circuit completed between the projections and the coating solution.

The amount of payload coated can be controlled using electrophoresis to draw all of the payload in the coating solution to the projections.

Insoluble compounds can be homogeneously distributed in the very viscous coating solution or coating gel for coating. Because of the extremely high viscosity of coating media, the insoluble compounds will not precipitate out from coating media. Therefore, they can be coated on the projection tips.

A number of further variations and options for use with the above described devices will now be described.

Herein, the terms “projection”, “micro-nanoprojection”, “nanoneedle”, “nanoprojection”, “needle”, “rod” etc are used interchangeably to describe the projections. Similarly, the terms dipping and immersion are also used interchangeably and refer to any situation in which projections are inserted into a coating solution.

A further feature is that the projections may be used for delivery not only through the skin but through other body surfaces, including mucosal surfaces, to cellular sites below the outer layer or layers of such surfaces. The term “internal site”, as used herein, is to be understood as indicating a site below the outer layer(s) of skin and other tissues for which the devices of the present invention are to be used.

The device is suitable for intracellular delivery. The device is suitable for delivery to specific organelles within cells. Examples of organelles to which the device can be applied include a cell nucleus, or endoplasmic reticulum, for example.

In one example the device is provided having a needle support section, that is to say the projections comprise a suitable support section, of sufficient length to reach the desired site and a (needle) delivery end section having a length no greater than 20 microns and a maximum width no greater than 5 microns, preferably no greater than 2 microns.

In one example, the maximum width of the delivery end section is no greater than 1000 nm, even more preferably the maximum width of the delivery end section is no greater than 500 nm.

In a further example, the device is for mucosal delivery. This device may have a needle support section, that is to say the projections comprise a suitable support section, of sufficient length to reach the desired site, such as of length at least 100 microns and a (needle) delivery end section having a length no greater than 20 microns and a maximum width no greater than 5 microns, preferably no greater than 2 microns.

In one example, the device of the invention is for delivery to lung, eye, cornea, sclera or other internal organ or tissue. In a further example, the device is for in-vitro delivery to tissue, cell cultures, cell lines, organs, artificial tissues and tissue engineered products.

This device typically has a needle support section, that is to say the projections comprise a suitable support section, of length at least 5 microns and a needle delivery end section having a length no greater than 20 microns and a maximum width no greater than 5 microns, preferably no greater than 2 microns.

In one example, the device comprises projections in which the (needle) delivery end section and support length, that is to say the “needle support section”, is coated with a bioactive material across the whole or part of its length. The (needle) delivery end section and support length may be coated on selective areas thereof. This may depend upon the bioactive material being used or the target selected for example.

In a further example, a bioactive material is incorporated into the material of which the needle, or projection, is composed such that it will be released on patch application. All, or part of the projection may be constructed of a biocompatible, biodegradable polymer (such as Poly Lactic Acid (PLA), PolyGlycolic Acid (PGA) or PGLA or Poly Glucleic Acid), which is formulated with the bioactive material of choice. The projections may then be inserted into the appropriate target site and, as they dissolve, the bioactive material will enter the organelle(s)/cells.

Examples of bioactive materials, which are not intended to be limiting with respect to the invention include polynucleotides and nucleic acid or protein molecules, antigens, allergens, adjuvants, molecules, elements or compounds. In addition, the device may be coated with materials such as biosensors, nanosensors or MEMS.

Illustrative material that can be delivered may include any or more of: small chemical or biochemical compounds including drugs, metabolites, amino acids, sugars, lipids, saponins, and hormones; macromolecules such as complex carbohydrates, phospholipids, peptides, polypeptides, peptidomimetics, and nucleic acids; or other organic (carbon containing) or inorganic molecules; and particulate matter including whole cells, bacteria, viruses, virus-like particles, cell membranes, dendrimers and liposomes.

The material can be selected from nucleic acids, illustrative examples of which include DNA, RNA, sense oligonucleotides, antisense oligonucleotides, ribozymes, small interfering oligonucleotides (siRNAs), micro RNAs (miRNAs), repeat associated RNAs (rasiRNA), effector RNAs (eRNAs), and any other oligonucleotides known in the art, which inhibit transcription and/or translation of a mutated or other detrimental protein. In illustrative examples of this type, the nucleic acid is in the form of an expression vector from which a polynucleotide of interest is expressible. The polynucleotide of interest may encode a polypeptide or an effector nucleic acid molecule such as sense or antisense oligonucleotides, siRNAs, miRNAs and eRNAs.

The material can be selected from peptides or polypeptides, illustrative examples of which include insulin, proinsulin, follicle stimulating hormone, insulin like growthfactor-1, insulin like growth factor-2, platelet derived growth factor, epidermal growth factor, fibroblast growth factors, nerve growth factor, colony stimulating factors, transforming growth factors, tumor necrosis factor, calcitonin, parathyroid hormone, growth hormone, bone morphogenic protein, erythropoietin, hemopoietic growth factors, luteinizing hormone, glucagon, glucagon likepeptide-1, anti-angiogenic proteins, clotting factors, anti-clotting factors, atrial natriuretic factor, plasminogen activators, bombesin, thrombin, enkephalinase, vascular endothelial growth factor, interleukins, viral antigens, non-viral antigens, transport proteins, and antibodies.

The material can be selected from receptor ligands. Illustrative examples of receptors include Fc receptor, heparin sulfate receptor, vitronectin receptor, Vcam-1 receptor, hemaglutinin receptor, Pvr receptor, Icam-1 receptor, decay-accelerating protein (CD55) receptor, Car (coxsackievirus-adenovirus) receptor, integrin receptor, sialic acid receptor, HAVCr-1 receptor, low-density lipoprotein receptor, BGP (biliary glycoprotien) receptor, aminopeptidease N receptor, MHC class-1 receptor, laminin receptor, nicotinic acetylcholine receptor, CD56 receptor, nerve growth factor receptor, CD46 receptor, asialoglycoprotein receptor Gp-2, alpha-dystroglycan receptor, galactosylceramide receptor, Cxcr4 receptor, Glvr1 receptor, Ram-1 receptor, Cat receptor, Tva receptor, BLVRcp1 receptor, MHC class-2 receptor, toll-like receptors (such as TLR-1 to -6) and complement receptors.

The material can be selected from antigens including endogenous antigens produced by a host that is the subject of the stimulus or material delivery or exogenous antigens that are foreign to that host. The antigens may be in the form of soluble peptides or polypeptides or polynucleotides from which an expression product (e.g., protein or RNA) is producible.

Suitable endogenous antigens include, but are not restricted to, cancer or tumor antigens. Non-limiting examples of cancer or tumor antigens include antigens from a cancer or tumor selected from ABL1 proto-oncogene, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, anal cancer, angiosarcoma, aplastic anemia, astrocytoma, ataxia-telangiectasia, basal cell carcinoma (skin), bladder cancer, bone cancers, bowel cancer, brain stem glioma, brain and CNS tumors, breast cancer, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, dermatofibrosarcoma protuberans, desmoplastic small round cell tumor, ductal carcinoma, endocrine cancers, endometrial cancer, ependymoma, oesophageal cancer, Ewing's Sarcoma, Extra-Hepatic Bile Duct Cancer, Eye Cancer, Eye: Melanoma, Retinoblastoma, Fallopian Tube cancer, Fanconi anemia, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynecological cancers, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's cell histiocytosis, laryngeal cancer, leiomyosarcoma, leukemia, Li-Fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid tumor of kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, Nijmegen breakage syndrome, non-melanoma skin cancer, non-small-cell-lung-cancer (NSCLC), ocular cancers, esophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal tumours, pituitary cancer, polycythemia vera, prostate cancer, rare cancers and associated disorders, renal cell carcinoma, retinoblastoma, rhabdomyosarcoma, Rothmund-Thomson syndrome, salivary gland cancer, sarcoma, schwannoma, Sezary syndrome, skin cancer, small cell lung cancer (SCLC), small intestine cancer, soft tissue sarcoma, spinal cord tumors, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis-/-ureter), trophoblastic cancer, urethral cancer, urinary system cancer, uroplakins, uterine sarcoma, uterus cancer, vaginal cancer, vulva cancer, Waldenstrom's macroglobulinemia, Wilms' tumor. In certain examples, the cancer or tumor relates to melanoma. Illustrative examples of melanoma-related antigens include melanocyte differentiation antigen (e.g., gp100, MART, Melan-A/MART-1, TRP-1, Tyros, TRP2, MC1R, MUC1F, MUC1R or a combination thereof) and melanoma-specific antigens (e.g., BAGE, GAGE-1, gp100In4, MAGE-1 (e.g., GenBank Accession No. X54156 and AA494311), MAGE-3, MAGE4, PRAME, TRP2IN2, NYNSO1a, NYNSO1b, LAGE1, p97 melanoma antigen (e.g., GenBank Accession No. M12154) p5 protein, gp75, oncofetal antigen, GM2 and GD2 gangliosides, cdc27, p21ras, gp 100^{Pme1117} or a combination thereof. Other tumour-specific antigens include, but are not limited to: etv6, am11, cyclophilin b (acute lymphoblastic leukemia); Ig-idiotype (B cell lymphoma); E-cadherin, α-catenin, β-catenin, γ-catenin, p 120ctn (glioma); p21ras (bladder cancer); p21ras (biliary cancer); MUC family, HER2/neu, c-erbB-2 (breast cancer); p53, p21ras (cervical carcinoma); p21ras, HER2/neu, c-erbB-2, MUC family, Cripto-1protein, Pim-1 protein (colon carcinoma); Colorectal associated antigen (CRC)-CO17-1A/GA733, APC (colorectal cancer); carcinoembryonic antigen (CEA) (colorectal cancer; choriocarcinoma); cyclophilin b (epithelial cell cancer); HER2/neu, c-erbB-2, ga733 glycoprotein (gastric cancer); α-fetoprotein (hepatocellular cancer); Imp-1, EBNA-1 (Hodgkin's lymphoma); CEA, MAGE-3, NY-ESO-1 (lung cancer); cyclophilin b (lymphoid cell-derived leukemia); MUC family, p21ras (myeloma); HER2/neu, c-erbB-2 (non-small cell lung carcinoma); Imp-1, EBNA-1 (nasopharyngeal cancer); MUC family, HER2/neu, c-erbB-2, MAGE-A4, NY-ESO-1 (ovarian cancer); Prostate Specific Antigen (PSA) and its antigenic epitopes PSA-1, PSA-2, and PSA-3, PSMA, HER2/neu, c-erbB-2, ga733 glycoprotein (prostate cancer); HER2/neu, c-erbB-2 (renal cancer); viral products such as human papillomavirus proteins (squamous cell cancers of the cervix and esophagus); NY-ESO-1 (testicular cancer); and HTLV-1 epitopes (T cell leukemia).

Foreign antigens are suitably selected from transplantation antigens, allergens as well as antigens from pathogenic organisms. Transplantation antigens can be derived from donor cells or tissues from e.g., heart, lung, liver, pancreas, kidney, neural graft components, or from the donor antigen-presenting cells bearing MHC loaded with self antigen in the absence of exogenous antigen.

Non-limiting examples of allergens include Fel d 1 (i.e., the feline skin and salivary gland allergen of the domestic cat Felis domesticus, the amino acid sequence of which is disclosed International Publication WO 91/06571), Der p I, Der p II, Der fI or Der fII (i.e., the major protein allergens from the house dust mite dermatophagoides, the amino acid sequence of which is disclosed in International Publication WO 94/24281). Other allergens may be derived, for example from the following: grass, tree and weed (including ragweed) pollens; fungi and moulds; foods such as fish, shellfish, crab, lobster, peanuts, nuts, wheat gluten, eggs and milk; stinging insects such as bee, wasp, and hornet and the chirnomidae (non-biting midges); other insects such as the housefly, fruitfly, sheep blow fly, screw worm fly, grain weevil, silkworm, honeybee, non-biting midge larvae, bee moth larvae, mealworm, cockroach and larvae of Tenibrio molitor beetle; spiders and mites, including the house dust mite; allergens found in the dander, urine, saliva, blood or other bodily fluid of mammals such as cat, dog, cow, pig, sheep, horse, rabbit, rat, guinea pig, mouse and gerbil; airborne particulates in general; latex; and protein detergent additives.

The material can be pathogenic organisms such as, but are not limited to, viruses, bacteria, fungi parasites, algae and protozoa and amoebae. Illustrative viruses include viruses responsible for diseases including, but not limited to, measles, mumps, rubella, poliomyelitis, hepatitis A, B (e.g., GenBank Accession No. E02707), and C (e.g., GenBank Accession No. E06890), as well as other hepatitis viruses, influenza, adenovirus (e.g., types 4 and 7), rabies (e.g., GenBank Accession No. M34678), yellow fever, Epstein-Barr virus and other herpesviruses such as papillomavirus, Ebola virus, influenza virus, Japanese encephalitis (e.g., GenBank Accession No. E07883), dengue (e.g., GenBank Accession No. M24444), hantavirus, Sendai virus, respiratory syncytial virus, othromyxoviruses, vesicular stomatitis virus, visna virus, cytomegalovirus and human immunodeficiency virus (HIV) (e.g., GenBank Accession No. U18552). Any suitable antigen derived from such viruses are useful in the practice of the present invention. For example, illustrative retroviral antigens derived from HIV include, but are not limited to, antigens such as gene products of the gag, pol, and env genes, the Nef protein, reverse transcriptase, and other HIV components. Illustrative examples of hepatitis viral antigens include, but are not limited to, antigens such as the S, M, and L proteins of hepatitis B virus, the pre-S antigen of hepatitis B virus, and other hepatitis, e.g., hepatitis A, B, and C, viral components such as hepatitis C viral RNA. Illustrative examples of influenza viral antigens include; but are not limited to, antigens such as hemagglutinin and neurarninidase and other influenza viral components. Illustrative examples of measles viral antigens include, but are not limited to, antigens such as the measles virus fusion protein and other measles virus components. Illustrative examples of rubella viral antigens include, but are not limited to, antigens such as proteins E1 and E2 and other rubella virus components; rotaviral antigens such as VP7sc and other rotaviral components. Illustrative examples of cytomegaloviral antigens include, but are not limited to, antigens such as envelope glycoprotein B and other cytomegaloviral antigen components. Non-limiting examples of respiratory syncytial viral antigens include antigens such as the RSV fusion protein, the M2 protein and other respiratory syncytial viral antigen components. Illustrative examples of herpes simplex viral antigens include, but are not limited to, antigens such as immediate early proteins, glycoprotein D, and other herpes simplex viral antigen components. Non-limiting examples of varicella zoster viral antigens include antigens such as 9PI, gpII, and other varicella zoster viral antigen components. Non-limiting examples of Japanese encephalitis viral antigens include antigens such as proteins E, M-E, M-E-NS 1, NS 1, NS 1-NS2A, 80% E, and other Japanese encephalitis viral antigen components. Representative examples of rabies viral antigens include, but are not limited to, antigens such as rabies glycoprotein, rabies nucleoprotein and other rabies viral antigen components. Illustrative examples of papillomavirus antigens include, but are not limited to, the L1 and L2 capsid proteins as well as the E6/E7 antigens associated with cervical cancers, See Fundamental Virology, Second Edition, eds. Fields, B. N. and Knipe, D. M., 1991, Raven Press, New York, for additional examples of viral antigens.

Illustrative examples of fungi include Acremonium spp., Aspergillus spp., Basidiobolus spp., Bipolaris spp., Blastomyces dermatidis, Candida spp., Cladophialophora carrionii, Coccoidiodes immitis, Conidiobolus spp., Cryptococcus spp., Curvularia spp., Epidermophyton spp., Exophiala jeanselmei, Exserohilum spp., Fonsecaea compacta, Fonsecaea pedrosoi, Fusarium oxysporum, Fusarium solani, Geotrichum candidum, Histoplasma capsulatum var. capsulatum, Histoplasma capsulatum var. duboisii, Hortaea werneckii, Lacazia loboi, Lasiodiplodia theobromae, Leptosphaeria senegalensis, Madurella grisea, Madurella mycetomatis, Malassezia furfur, Microsporum spp., Neotestudina rosatii, Onychocola canadensis, Paracoccidioides brasiliensis, Phialophora verrucosa, Piedraia hortae, Piedra iahortae, Pityriasis versicolor, Pseudallesheria boydii, Pyrenochaeta romeroi, Rhizopus arrhizus, Scopulariopsis brevicaulis, Scytalidium dimidiatum, Sporothrix schenckii, Trichophyton spp., Trichosporon spp., Zygomcete fungi, Absidia corymbifera, Rhizomucor pusillus and Rhizopus arrhizus. Thus, representative fungal antigens that can be used in the compositions and methods of the present invention include, but are not limited to, candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Illustrative examples of bacteria include bacteria that are responsible for diseases including, but not restricted to, diphtheria (e.g., Corynebacterium diphtheria), pertussis (e.g., Bordetella pertussis, GenBank Accession No. M35274), tetanus (e.g., Clostridium tetani, GenBank Accession No. M64353), tuberculosis (e.g., Mycobacterium tuberculosis), bacterial pneumonias (e.g., Haemophilus influenzae.), cholera (e.g., Vibrio cholerae), anthrax (e.g., Bacillus anthracis), typhoid, plague, shigellosis (e.g., Shigella dysenteriae), botulism (e.g., Clostridium botulinum), salmonellosis (e.g., GenBank Accession No. L03833), peptic ulcers (e.g., Helicobacter pylori), Legionnaire's Disease, Lyme disease (e.g., GenBank Accession No. U59487), Other pathogenic bacteria include Escherichia coli, Clostridium perfringens, Pseudomonas aeruginosa, Staphylococcus aureus and Streptococcus pyogenes. Thus, bacterial antigens which can be used in the compositions and methods of the invention include, but are not limited to: pertussis bacterial antigens such as pertussis toxin, filamentous hemagglutinin, pertactin, F M2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components, streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components, pneumococcal bacterial antigens such as pneumolysin, pneumococcal capsular polysaccharides and other pnermiococcal bacterial antigen components; Haemophilus influenza bacterial antigens such as capsular polysaccharides and other Haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens.

Illustrative examples of protozoa include protozoa that are responsible for diseases including, but not limited to, malaria (e.g., GenBank Accession No. X53832), hookworm, onchocerciasis (e.g., GenBank Accession No. M27807), schistosomiasis (e.g., GenBank Accession No. LOS 198), toxoplasmosis, trypanosomiasis, leishmaniasis, giardiasis (GenBank Accession No. M33641), amoebiasis, filariasis (e.g., GenBank Accession No. J03266), borreliosis, and trichinosis. Thus, protozoal antigens which can be used in the compositions and methods of the invention include, but are not limited to: plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components.

The material can be toxin components acting as antigens. Illustrative examples of toxins include, but are not restricted to, staphylococcal enterotoxins, toxic shock syndrome toxin; retroviral antigens (e.g., antigens derived from HIV), streptococcal antigens, staphylococcal enterotoxin-A (SEA), staphylococcal enterotoxin-B (SEB), staphylococcal enterotoxin₁₋₃ (SE₁₋₃), staphylococcal enterotoxin-D (SED), staphylococcal enterotoxin-E (SEE) as well as toxins derived from mycoplasma, mycobacterium, and herpes viruses.

In specific examples, the antigen is delivered to antigen-presenting cells. Such antigen-presenting cells include professional or facultative antigen-presenting cells. Professional antigen-presenting cells function physiologically to present antigen in a form that is recognised by specific T cell receptors so as to stimulate or anergise a T lymphocyte or B lymphocyte mediated immune response. Professional antigen-presenting cells not only process and present antigens in the context of the major histocompatability complex (MHC), but also possess the additional immunoregulatory molecules required to complete T cell activation or induce a tolerogenic response. Professional antigen-presenting cells include, but are not limited to, macrophages, monocytes, B lymphocytes, cells of myeloid lineage, including monocytic-granulocytic-DC precursors, marginal zone Kupffer cells, microglia, T cells, Langerhans cells and dendritic cells including interdigitating dendritic cells and follicular dendritic cells. Non-professional or facultative antigen-presenting cells typically lack one or more of the immunoregulatory molecules required to complete T lymphocyte activation or anergy. Examples of non-professional or facultative antigen-presenting cells include, but are not limited to, activated T lymphocytes, eosinophils, keratinocytes, astrocytes, follicular cells, microglial cells, thymic cortical cells, endothelial cells, Schwann cells, retinal pigment epithelial cells, myoblasts, vascular smooth muscle cells, chondrocytes, enterocytes, thymocytes, kidney tubule cells and fibroblasts. In some examples, the antigen-presenting cell is selected from monocytes, macrophages, B lymphocytes, cells of myeloid lineage, dendritic cells or Langerhans cells. In certain advantageous examples, the antigen-presenting cell expresses CD11c and includes a dendritic cell or Langerhans cell. In some examples the antigen-presenting cell stimulates an immune response. In other examples, the antigen-presenting cell induces a tolerogenic response.

The delivery of exogenous antigen to an antigen-presenting cell can be enhanced by methods known to practitioners in the art. For example, several different strategies have been developed for delivery of exogenous antigen to the endogenous processing pathway of antigen-presenting cells, especially dendritic cells. These methods include insertion of antigen into pH-sensitive liposomes (Zhou and Huang, 1994, Immunomethods, 4:229-235), osmotic lysis of pinosomes after pinocytic uptake of soluble antigen (Moore et al., 1988, Cell, 54:777-785), coupling of antigens to potent adjuvants (Aichele et al., 1990, J. Exp. Med., 171: 1815-1820; Gao et al., 1991, J. Immunol., 147: 3268-3273; Schulz et al., 1991, Proc. Natl. Acad Sci. USA, 88: 991-993; Kuzu et al., 1993, Euro. J. Immunol., 23: 1397-1400; and Jondal et al., 1996, Immunity 5: 295-302) and apoptotic cell delivery of antigen (Albert et al. 1998, Nature 392:86-89; Albert et al. 1998, Nature Med. 4:1321-1324; and in International Publications WO 99/42564 and WO 01/85207). Recombinant bacteria (eg. E. coli) or transfected host mammalian cells may be pulsed onto dendritic cells (as particulate antigen, or apoptotic bodies respectively) for antigen delivery. Recombinant chimeric virus-like particles (VLPs) have also been used as vehicles for delivery of exogenous heterologous antigen to the MHC class I processing pathway of a dendritic cell line (Bachmann et al., 1996, Eur. J. Immunol., 26(11): 2595-2600).

Alternatively, or in addition, an antigen may be linked to, or otherwise associated with, a cytolysin to enhance the transfer of the antigen into the cytosol of an antigen-presenting cell of the invention for delivery to the MHC class I pathway. Exemplary cytolysins include saponin compounds such as saponin-containing Immune Stimulating Complexes (ISCOMs) (see e.g., Cox and Coulter, 1997, Vaccine 15(3): 248-256 and U.S. Pat. No. 6,352,697), phospholipases (see, e.g., Camilli et al., 1991, J. Exp. Med. 173: 751-754), pore-forming toxins (e.g., an α-toxin), natural cytolysins of gram-positive bacteria, such as listeriolysin O (LLO, e.g., Mengaud et al., 1988, Infect. Immun. 56: 766-772 and Portnoy et al., 1992, Infect. Immun. 60: 2710-2717), streptolysin O (SLO, e.g., Palmer et al., 1998, Biochemistry 37(8):2378-2383) and perfringolysin O (PFO, e.g., Rossjohn et al., Cell 89(5): 685-692). Where the antigen-presenting cell is phagosomal, acid activated cytolysins may be advantageously used. For example, listeriolysin exhibits greater pore-forming ability at mildly acidic pH (the pH conditions within the phagosome), thereby facilitating delivery of vacuole (including phagosome and endosome) contents to the cytoplasm (see, e.g., Portnoy et al., Infect. Immun. 1992, 60: 2710-2717).

The cytolysin may be provided together with a pre-selected antigen in the form of a single composition or may be provided as a separate composition, for contacting the antigen-presenting cells. In one example, the cytolysin is fused or otherwise linked to the antigen, wherein the fusion or linkage permits the delivery of the antigen to the cytosol of the target cell. In another example, the cytolysin and antigen are provided in the form of a delivery vehicle such as, but not limited to, a liposome or a microbial delivery vehicle selected from virus, bacterium, or yeast. Suitably, when the delivery vehicle is a microbial delivery vehicle, the delivery vehicle is non-virulent. In a preferred example of this type, the delivery vehicle is a non-virulent bacterium, as for example described by Portnoy et al. in U.S. Pat. No. 6,287,556, comprising a first polynucleotide encoding a non-secreted functional cytolysin operably linked to a regulatory polynucleotide which expresses the cytolysin in the bacterium, and a second polynucleotide encoding one or more pre-selected antigens. Non-secreted cytolysins may be provided by various mechanisms, e.g., absence of a functional signal sequence, a secretion incompetent microbe, such as microbes having genetic lesions (e.g., a functional signal sequence mutation), or poisoned microbes, etc. A wide variety of nonvirulent, non-pathogenic bacteria may be used; preferred microbes are relatively well characterised strains, particularly laboratory strains of E. coli, such as MC4100, MC1061, DH5α, etc. Other bacteria that can be engineered for the invention include well-characterised, nonvirulent, non-pathogenic strains of Listeria monocytogenes, Shigella flexneri, mycobacterium, Salmonella, Bacillus subtilis, etc. In a particular example, the bacteria are attenuated to be non-replicative, non-integrative into the host cell genome, and/or non-motile inter- or intra-cellularly.

The coated patches described above can be used to deliver one or more antigens to virtually any antigen-presenting cell capable of endocytosis of the subject vehicle, including phagocytic and non-phagocytic antigen-presenting cells. In examples when the delivery vehicle is a microbe, the subject methods generally require microbial uptake by the target cell and subsequent lysis within the antigen-presenting cell vacuole (including phagosomes and endosomes).

In other examples, the antigen is produced inside the antigen-presenting cell by introduction of a suitable expression vector as for example described above. The antigen-encoding portion of the expression vector may comprise a naturally-occurring sequence or a variant thereof, which has been engineered using recombinant techniques. In one example of a variant, the codon composition of an antigen-encoding polynucleotide is modified to permit enhanced expression of the antigen in a target cell or tissue of choice using methods as set forth in detail in International Publications WO 99/02694 and WO 00/42215. Briefly, these methods are based on the observation that translational efficiencies of different codons vary between different cells or tissues and that these differences can be exploited, together with codon composition of a gene, to regulate expression of a protein in a particular cell or tissue type. Thus, for the construction of codon-optimised polynucleotides, at least one existing codon of a parent polynucleotide is replaced with a synonymous codon that has a higher translational efficiency in a target cell or tissue than the existing codon it replaces. Although it is preferable to replace all the existing codons of a parent nucleic acid molecule with synonymous codons which have that higher translational efficiency, this is not necessary because increased expression can be accomplished even with partial replacement. Suitably, the replacement step affects 5, 10, 15, 20, 25, 30%, more preferably 35, 40, 50, 60, 70% or more of the existing codons of a parent polynucleotide.

The expression vector for introduction into the antigen-presenting cell will be compatible therewith such that the antigen-encoding polynucleotide is expressible by the cell. For example, expression vectors of this type can be derived from viral DNA sequences including, but not limited to, adenovirus, adeno-associated viruses, herpes-simplex viruses and retroviruses such as B, C, and D retroviruses as well as spumaviruses and modified lentiviruses. Suitable expression vectors for transfection of animal cells are described, for example, by Wu and Ataai (2000, Curr. Opin. Biotechnol. 11(2):205-208), Vigna and Naldini (2000, J. Gene Med 2(5):308-316), Kay, et al. (2001, Nat. Med 7(1):33-40), Athanasopoulos, et al. (2000, Int. J. Mol. Med. 6(4):363-375) and Walther and Stein (2000, Drugs 60(2):249-271).

In one aspect, the device is provided in the form of a patch containing a plurality of needles (projections) for application to a body surface. A multiplicity of projections can allow multiple cells and organelles to be targeted and provided with a material at the same time.

The patch may be of any suitable shape, such as square or round for example. The overall number of projections per patch depends upon the particular application in which the device is to be used. Preferably, the patch has at least 10 needles per mm, and more preferably at least 100 needles per mm². Considerations and specific examples of such a patch are provided in more detail below.

Examples of specific manufacturing steps used to fabricate the device are described in greater detail below. In one preferred aspect, the device of the invention is constructed from biocompatible materials such as Titanium, Gold, Silver or Silicon, for example. This may be the entire device, or alternatively it may only be the projections or the delivery end section of the projections which are made from the biocompatible materials.

One manufacturing method for the device utilises the Deep Reactive Ion Etching (DRIE) of the patterns direct from silicon wafers, see the construction section below.

Another manufacturing method for the device utilises manufacturing from a male template constructed with X-ray lithography, electrodeposition and moulding (LIGA). The templates are then multiply inserted into a soft polymer to produce a plurality of masks. The masks are then vacuum deposited/sputtered with the material of choice for the nanoprojections, such as titanium, gold, silver, or tungsten. Magnetron sputtering may also be applied, see the construction section below.

An alternative means for producing masks is with 2 photon Stereolithography, a technique which is known in the art and is described in more detail below.

In one example, the device is constructed of silicon.

The device may be for a single use or may be used and then recoated with the same or a different bioactive material or other stimulus, for example.

In one example, the device comprises projections which are of differing lengths and/or diameters (or thicknesses depending on the shape of the projections) to allow targeting of different targets within the same use of the device.

Persons skilled in the art will appreciate that numerous variations and modifications will become apparent. All such variations and modifications which become apparent to persons skilled in the art, should be considered to fall within the spirit and scope that the invention broadly appearing before described.

1. A method of coating projections on a patch, the method including:
a) selecting a coating solution viscosity, the viscosity being selected to reduce the degree of capillary action between the patch and the coating solution; and,
b) immersing at least part of tips of projections in a coating solution having the selected coating solution viscosity such that substantially only tips of the projections are coated.

2. A method according to claim 1, wherein the coating solution viscosity is at least one of:
a) 1 Pa.S;
b) 10 Pa.S; and,
c) 50 Pa.S.

3. A method according to claim 1, wherein the method includes:
a) selecting the coating solution viscosity in accordance with an immersion time; and,
b) immersing at least part of the tips of the projections for the immersion time.

4. A method according to claim 3, wherein the immersion time is less than at least one of:
a) 60 minutes;
b) 10 minutes;
c) 1 minute; and,
d) 10 seconds.

5. A method according to claim 1, wherein the method includes drying the coated projection tips.

6. A method according to claim 5, wherein the method includes drying the coated projection tips using at least one of:
a) exposure to vacuum;
b) temperature control;
c) humidity control;
d) a gas flow.

7. A method according to claim 1, wherein viscosity is selected in accordance with patch properties including at least one of:
a) projection size;
b) projection shape; and,
c) projection spacing.

8. A method according to claim 1, wherein the viscosity is selected in accordance with a contact angle representing hydrophilicity or hydrophobicity of the patch.

9. A method according to claim 1, wherein the method includes modifying the surface properties of the patch to thereby control at least one of:
a) hydrophilicity of the patch;
b) hydrophobocity of the patch; and,
c) wettability of the patch.

10. A method according to claim 9, wherein the method includes modifying the surface properties of the patch prior to immersing the tips.

11. A method according to claim 9, wherein the method includes modifying the surface properties of the patch by modifying a surface structure of at least part of the patch.

12. A method according to claim 11, wherein the surface structure includes a surface roughness.

13. A method according to claim 11, wherein the method includes modifying the surface structure by at least one of:
a) mechanical means; and,
b) chemical means.

14. A method according to claim 9, wherein the method includes modifying the surface properties of the patch by coating the patch.

15. A method according to claim 14, wherein the method includes coating the patch with at least one of:
a) 3-aminopropyl triethoxysilane (3-APTES) solution; and,
b) Methylcellulose.

16. A method according to claim 1, wherein the method includes selecting a coating solution surface tension.

17. A method according to claim 1, wherein the method includes selecting at least the viscosity to thereby control an amount of coating on the tips.

18. A method according to claim 1, wherein the coating solution includes a material that is insoluble in the coating solution and wherein the material is distributed substantially homogenously throughout the coating solution.

19. A method according to claim 18, wherein the material is at least one of:
a) a biological agent; and,
b) a therapeutic agent.

20. A method according to claim 18, wherein the material is at least one of:
a) nanoparticles;
b) a nucleic acid or protein;
c) an antigen, allergen, or adjuvant;
d) parasites, bacteria, viruses, or virus-like particles;
e) quantum dots, SERS tags, raman tags or other nanobiosensors;
f) metals or metallic compounds;
g) molecules, elements or compounds;
h) DNA having a concentration of between 0.01 mg/ml and 5 mg/ml; and,
i) protein having a concentration of between 0.01 mg/ml and 50 mg/ml

21. A method according to claim 1, wherein the coating solution includes at least one of:
a) a viscosity enhancer;
b) a detergent;
c) a surfactant; and,
d) an adjuvant.

22. A method according to claim 21, wherein the adjuvant acts as a detergent.

23. A method according to claim 21, wherein at least one of:
a) the viscosity enhancer is 0% to 90% of the coating solution; and,
b) the detergent is 0% to 90% of the coating solution.

24. A method according to claim 21, wherein the viscosity enhancer is at least one of:
a) honey;
b) pectin;
c) methylcellulose;
d) carboxymethylcellulose (CMC);
e) sodium alginate;
f) gelatine;
g) agar; and,
h) agarose.

25. A method according to claim 1, wherein the method includes:
a) applying an electrical signal to the coating solution and the projections; and,
b) controlling the coating process using the electrical signal.

26. A method according to claim 1, wherein the method includes applying an electrical signal to the coating solution and the projections to thereby attract a material within the coating solution onto the projections using electrophoresis.

27. A method according to claim 1, wherein the method includes controlling a length of coating on the projections by controlling a depth of immersion in the coating solution.

28. A method according to claim 27, wherein the method includes controlling a depth of immersion in the coating solution based on a coating solution depth.

29. A method according to claim 1, wherein the method includes immersing the tips by placing at least the projections in a well containing the coating solution.

30. A method according to claim 29, wherein the well includes a stop, and wherein the stop cooperates with the patch such that only projection tips are immersed in the coating solution.

31. A method according to claim 30, wherein the stop abuts against a patch base.

32. A method according to claim 29, wherein the projection tips abut against a floor of the well.

33. A method according to claim 1, wherein the method includes coating the projections a number of times.

34. A method according to claim 33, wherein the method includes:
a) coating the surface a first time using a first set of coating parameters; and,
b) coating the surface at least a second time using a second set of coating parameters different to the first set of coating parameters.

35. A method according to claim 33, wherein the method includes coating the projections a number of times to thereby provide at least first and second coating layers.

36. A method according to claim 35, wherein the second layer overlays the first layer to thereby protect the first layer during insertion into the subject.

37. A method according to claim 35, wherein the first and second layers include different coating materials.

38. A method according to claim 35, wherein the method includes coating a first length of the projection using a first coating material and a second length of the projection using a second coating material.

39. A method according to claim 38, wherein the first and second coating lengths are selected to deliver material to a selected region in a subject.

40. A method according to claim 1, wherein the projections are solid.

41. A method according to claim 1, wherein the projections are non-porous and non-hollow.

42. A method of coating projections on a patch, the method including immersing at least part of tips of projections in a coating solution, the coating solution having a viscosity of greater than approximately 1 Pa.S.

43. Apparatus for use in coating projections on a patch, the apparatus including a positioning system for immersing at least part of tips of projections in a coating solution, the coating solution having a viscosity selected in accordance with patch properties so as to reduce the degree of capillary action between the patch and the coating solution such that substantially only tips of the projections are coated.

44. Apparatus according to claim 43, wherein the positioning system includes a support having an arm for supporting the patch relative to the coating solution.

45. Apparatus according to claim 44, wherein the arm is movable to thereby allow the relative position of the patch and the coating solution to be controlled.

46. Apparatus according to claim 45, wherein the positioning system includes a movable platform for supporting the coating solution to thereby allow the relative position of the patch and the coating solution to be controlled.

47. Apparatus according to claim 43, wherein the apparatus includes a controller for controlling the positioning system.

48. Apparatus according to claim 47, wherein the controller is coupled to a sensor for determining at least if the projection tips are immersed.

49. Apparatus according to claim 48, wherein the sensor is an imaging system for imaging the projections and the coating solution.

50. Apparatus according to claim 49, wherein the sensor includes a signal generator for:
a) applying an electrical signal to the projections and the coating solution; and,
b) providing an indication relating to the signal to the controller, thereby allowing the controller to determine if the projection tips are immersed.

51. Apparatus according to claim 43, wherein the apparatus includes a signal generator for applying an electrical signal to the projections and the coating solution to thereby attract a material within the coating solution onto the projections using electrophoresis.

52. Apparatus according to claim 43, wherein the apparatus is for controlling a length of coating on the projections by controlling a depth of immersion in the coating solution.

53. A method according to claim 52, wherein the method includes controlling a depth of immersion in the coating solution based on a coating solution depth.

54. Apparatus according to claim 43, wherein the apparatus includes a well containing the coating solution.

55. Apparatus according to claim 54, wherein the positioning system includes a stop provided on the well, and wherein the stop cooperates with the patch such that only projection tips are immersed in the coating solution.

56. Apparatus according to claim 55, wherein the stop abuts against a patch base.

57. Apparatus according to claim 56, wherein the positioning system includes the depth of coating solution in the well so that the projection tips abut against a floor of the well when the projection tips are immersed.

58. Apparatus according to claim 57, wherein the apparatus includes a force sensitive device for detecting if the projections are in contact with the well.

59. Apparatus according to claim 54, wherein a length of coating on the projections is controlled at least in part by a depth of coating solution in the well.

60. Apparatus according to claim 43, wherein the apparatus is for agitating the patch relative to the coating solution.

61. Apparatus according to claim 60, wherein the apparatus agitates the patch relative to the coating solution using at least one of:
a) an arm; and,
b) a movable platform

62. Apparatus according to claim 60, wherein the apparatus is for oscillating the patch relative to the coating solution.

63. Apparatus according to claim 62, wherein the apparatus includes a controller for controlling at least one of an amplitude and frequency of the oscillation.

64. Apparatus according to claim 63, wherein at least one of:
a) the amplitude of the oscillation is in the range of 0.01 to 100 μm; and,
b) the frequency of the oscillation is in the range of 1 to 10,000 Hz.

65. Apparatus according to claim 63, wherein at least one of:
a) the amplitude of the oscillation is approximately ±1 μm; and,
b) the frequency of the oscillation is approximately 400 Hz.

66. A patch for delivering material to a subject, the patch including a number of projections thereon, the projections being coated by immersion of at least part of the projections in a coating solution having a viscosity selected to reduce the degree of capillary action between the patch and the coating solution.

67. A patch according to claim 66, wherein the coating solution has a viscosity of at least:
a) 1 Pa.S;
b) 10 Pa.S; and,
c) 50 Pa.S.
